(19) United States
(12) Patent Application Publication
(10) Pub. No.: US 2024/0242842 A1
(43) Pub. Date: Jul. 18, 2024

(54) INFORMATION PROCESSING APPARATUS, INFORMATION PROCESSING METHOD, AND PROGRAM

(71) Applicant: SONY GROUP CORPORATION, TOKYO (JP)

(72) Inventors: MAKI IMOTO, TOKYO (JP); HARUKA KUTSUKI, TOKYO (JP); AKANE KONDO, TOKYO (JP)

(21) Appl. No.: 18/559,138

(22) PCT Filed: Jan. 13, 2022
(86) PCT No.: PCT/JP2022/000894

§ 371 Date: Nov. 6, 2023

(30) Foreign Application Priority Data
May 17, 2021 (JP) 2021-083276

(51) Int. Cl. G16H 50/30; A63B 24/00; G06T 7/70; G06V 40/20

(52) U.S. Cl. CPC G16H 50/30; A63B 24/0003; A63B 24/0075; G06T 7/70; G06V 40/20; G06T 2207/30242

(57) ABSTRACT

Provided are an information processing apparatus, an information processing method, and a program capable of promoting a better life by detecting and feeding back an action of a user. An information processing apparatus including a control unit that performs: a process of recognizing a user existing in a space on the basis of a detection result of a sensor disposed in the space and calculating health points indicating that a healthy behavior has been performed from an action of the user; and a process of giving notification of the health points.

### TECHNICAL FIELD

The present disclosure relates to an information processing apparatus, an information processing method, and a program.

### BACKGROUND ART

In order to live a good life, it is important to pay attention to moving the body in daily life. In recent years, it has been performed to wear a smart device such as a smartphone or a smart band on a daily basis, and to grasp one's exercise amount by looking at an activity amount such as the number of steps detected by the smart device.

Furthermore, Patent Document 1 below discloses a technique for continuing an action effective for maintaining health by granting points according to a measurement value of a wearable activity meter and enabling exchange of the point with a product or a service.

### CITATION LIST

### Patent Document

PATENT DOCUMENT 1: Japanese Patent Application Laid-Open No. 2003-141260

### SUMMARY OF THE INVENTION

### Problems to be Solved by the Invention

However, in the conventional technique, it is necessary to always wear the activity meter, which may not be preferable in a relaxed space such as a home.

Therefore, the present disclosure proposes an information processing apparatus, an information processing method, and a program capable of promoting a better life by detecting and feeding back an action of a user.

### Solutions to Problems

According to the present disclosure, there is proposed an information processing apparatus including a control unit that performs: a process of recognizing a user existing in a space on the basis of a detection result of a sensor disposed in the space and calculating health points indicating that a healthy behavior has been performed from an action of the user; and a process of giving notification of the health points.

According to the present disclosure, there is proposed an information processing method in which a processor including: recognizing a user existing in a space on the basis of a detection result of a sensor disposed in the space and calculating health points indicating that a healthy behavior has been performed from an action of the user; and giving notification of the health points.

According to the present disclosure, there is proposed a program for causing a computer to function as a control unit that performs: a process of recognizing a user existing in a space on the basis of a detection result of a sensor disposed in the space and calculating health points indicating that a healthy behavior has been performed from an action of the user; and a process of giving notification of the health points.

### BRIEF DESCRIPTION OF DRAWINGS

FIG. 1 is a diagram illustrating an overview of a system according to an embodiment of the present disclosure.

FIG. 2 is a diagram for explaining various functions according to the present embodiment.

FIG. 3 is a block diagram illustrating an example of a configuration of an information processing apparatus according to the present embodiment.

FIG. 4 is a flowchart illustrating an example of a flow of entire operation processing for implementing various functions according to the present embodiment.

FIG. 5 is a block diagram illustrating an example of a configuration of an information processing apparatus that implements a health point notification function according to a first example.

FIG. 6 is a diagram illustrating an example of notification contents according to a degree of interest in exercise according to the first example.

FIG. 7 is a flowchart illustrating an example of a flow of health point notification processing according to the first example.

FIG. 8 is a diagram illustrating an example of a health point notification to a user according to the first example.

FIG. 9 is a diagram illustrating an example of a health point notification to a user according to the first example.

FIG. 10 is a diagram illustrating an example of a health point confirmation screen according to the first example.

FIG. 11 is a block diagram illustrating an example of a configuration of an information processing apparatus that realizes a space production function according to a second example.

FIG. 12 is a flowchart illustrating an example of a flow of space production processing according to the second example.

FIG. 13 is a flowchart illustrating an example of a flow of space production processing during eating and drinking according to the second example.

FIG. 14 is a diagram illustrating an example of a video for space production according to the number of people during eating and drinking according to the second example.

FIG. 15 is a diagram for explaining imaging performed in response to a cheers action according to the second example.

FIG. 16 is a diagram for explaining an example of various types of output control performed in space production during eating and drinking according to the second example.

FIG. 17 is a block diagram illustrating an example of a configuration of an information processing apparatus that implements an exercise program providing function according to a third example.

FIG. 18 is a flowchart illustrating an example of a flow of exercise program providing processing according to the third example.

FIG. 19 is a flowchart illustrating an example of a flow of yoga program providing processing according to the third example.

FIG. 20 is a diagram illustrating an example of a screen of a yoga program according to the third example.

FIG. 21 is a diagram illustrating an example of a screen on which health points granted to a user by an end of the yoga program according to the third example is displayed.

### MODE FOR CARRYING OUT THE INVENTION

Hereinafter, preferred embodiments of the present disclosure will be described in detail with reference to the accompanying drawings. Note that, in the present specification and the drawings, components having substantially the same functional configuration are denoted by the same reference signs, and redundant explanations are omitted.

Furthermore, the description is given in the following order.
1. Overview
2. Configuration example
3. Operation processing
4. First example (Health point notification function)
4-1. Configuration example
4-2. Operation processing
4-3. Modified example
5. Second example (Space production function)
5-1. Configuration example
5-2. Operation processing
5-3. Modified example
6. Third example (Exercise program providing function)
6-1. Configuration example
6-2. Operation processing
6-3. Modified example
7. Supplement

### 1. Overview

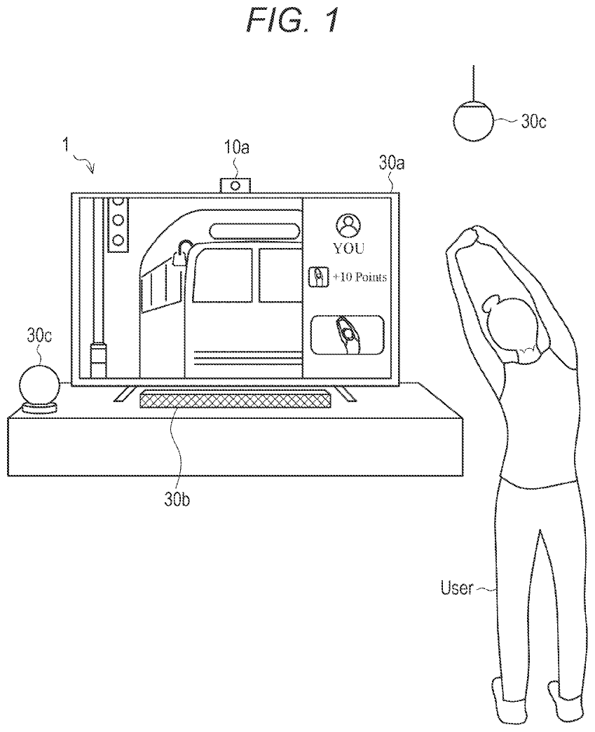

An overview of a system according to an embodiment of the present disclosure will be described with reference to FIG. 1. The system according to the present embodiment can promote a better life by detecting an action of a user and appropriately performing feedback.

FIG. 1 is a diagram illustrating an overview of a system according to an embodiment of the present disclosure. As illustrated in FIG. 1, a camera 10a that is an example of a sensor is disposed in a space. Furthermore, a display unit 30a that is an example of an output device that performs feedback is disposed in the space. The display unit 30a may be, for example, a home television receiver.

The camera 10a is attached to the display unit 30a, for example, and detects information regarding one or more persons existing around the display unit 30a. In a case where the display unit 30a is realized by a television receiver, the television receiver is usually installed at a relatively easily viewable position in a room, and thus it is possible to image the entire room by attaching the camera 10a to the display unit 30a. More specifically, the camera 10a continuously images the surroundings. As a result, the camera 10a according to the present embodiment can detect daily behavior of the user in the room including while the user is watching television.

Note that the output device that performs feedback is not limited to the display unit 30a, and may be, for example, a speaker 30b of the television receiver or a lighting device 30c installed in a room as illustrated in FIG. 1. There may be a plurality of the output devices. Furthermore, an arrangement place of each output device is not particularly limited. In the example illustrated in FIG. 1, the camera 10a is provided in an upper center of the display unit 30a, but may be provided in a lower center, may be provided in another place of the display unit 30a, or may be provided around the display unit 30a.

An information processing apparatus 1 according to the present embodiment performs control to recognize a user on the basis of a detection result (captured image) by the camera 10a, calculate health points indicating that a healthy behavior has been performed from an action of the user, and notify the user of the calculated health points. As illustrated in FIG. 1, for example, the notification may be performed from the display unit 30a. The healthy behavior is a predetermined posture or movement registered in advance. More specifically, examples thereof include various kinds of stretching, muscle strength training, exercise, walking, laughing, dancing, and housework.

As described above, in the present embodiment, stretching or the like performed casually while staying in the room is grasped as a numerical value such as a health point and fed back (given in notification) to the user, so that the user can naturally be conscious of exercise. Furthermore, since the user's action is detected by an external sensor, it is not necessary for the user to always wear a device such as an activity meter, and a burden on the user is reduced. The present system can also be implemented in a case where the user is in a relaxed space, allowing the user to be interested in exercise without placing a burden on the user, and promoting a healthy and better life.

Note that the information processing apparatus 1 according to the present embodiment may be implemented by a television receiver.

Furthermore, the information processing apparatus 1 according to the present embodiment may calculate, according to the health points of each user, an interest degree of the user in exercise, and determine notification contents according to the degree of interest in exercise. For example, in the notification to the user having a low degree of interest in exercise, the exercise may be promoted by giving a simple stretch proposal together.

Furthermore, the information processing apparatus 1 according to the present embodiment may acquire the context (situation) of the user on the basis of the detection result (captured image) by the camera 10a, and may give notification of the health points, for example, at a timing at which the information processing apparatus 1 does not disturb the viewing of content.

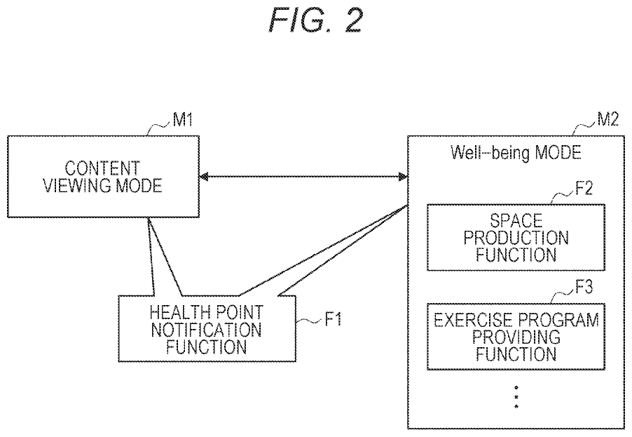

Furthermore, in the present system, by using the sensor (camera 10a) described with reference to FIG. 1 and the output device (display unit 30a or the like) that performs feedback, in addition to the function of giving notification of the health points described above, various functions for promoting a better life are realized. Hereinafter, a description will be made with reference to FIG. 2.

FIG. 2 is a diagram for explaining various functions according to the present embodiment. First, in a case where the information processing apparatus 1 is implemented by a display device used for viewing content such as a television receiver, switching between a content viewing mode M1 and a Well-being mode M2 can be performed as an operation mode of the information processing apparatus 1.

The content viewing mode M1 is an operation mode mainly intended to view content. The content viewing mode M1 can also be said to be an operation mode including, for example, a mode in a case where the information processing apparatus 1 (display device) is used as a conventional TV apparatus. In the content viewing mode M1, video and audio are displayed by receiving radio waves of television broadcasting, recorded television programs are displayed, and content distributed on the Internet such as a video distribution service is displayed. Furthermore, the information processing apparatus 1 (display device) may also be used as a monitor of a game device, and a game screen can be displayed in the content viewing mode M1. In the present embodiment, the “health point notification function F1” that is one of functions for promoting a better life can be implemented even during the content viewing mode M1.

On the other hand, “Well-being” is a concept meaning being in a physically, mentally, or socially good state (satisfied state), and can be also referred to as “happiness”. In the present embodiment, a mode mainly providing various functions for promoting a better life is referred to as a “Well-being mode”. In the “Well-being mode”, functions that lead to human body and mental health, such as personal health, hobbies, communication with people, and sleep, are provided. More specifically, for example, there are a space production function F2 and an exercise program providing function F3. Note that the “health point notification function F1” can also be implemented in the “Well-being mode”.

The transition from the content viewing mode M1 to the Well-being mode M2 may be performed by an explicit operation by the user, or may be automatically performed according to the user's situation (context). Examples of the explicit operation include a pressing operation of a predetermined button (Well-being button) provided in a remote controller used for operating the information processing apparatus 1 (display device). Furthermore, examples of the automatic transition according to the context include a case where one or more users present around the information processing apparatus 1 (display device) do not look at the information processing apparatus 1 (display device) for a certain period of time, a case where the users concentrate on things other than content viewing, and the like. After the transition to the Well-being mode M2, first, the screen moves to a home screen of the Well-being mode. From there, the mode transitions to each application (function) in the Well-being mode according to the context of the user. For example, in a case where one or more users are eating and drinking or are about to fall asleep, the information processing apparatus 1 performs the space production function F2 for outputting information such as a video, music, or lighting for corresponding space production. Furthermore, for example, in a case where one or more users actively perform some exercise, the information processing apparatus 1 determines the exercise that the user intends to perform and implements the exercise program providing function F3 that generates and provides an exercise program suitable for the users. As an example, for example, in a case where the user places a yoga mat, the information processing apparatus 1 generates and provides a yoga program suitable for the user.

As described above, in the information processing apparatus 1 (display device), by providing useful functions close to daily life even while content is not viewed, it is also possible to widen a range of use of the display device mainly used for content viewing.

The overview of the system according to the present embodiment has been described above. Next, a basic configuration example and operation processing of the information processing apparatus 1 included in the present system will be sequentially described.

### 2. Configuration Example

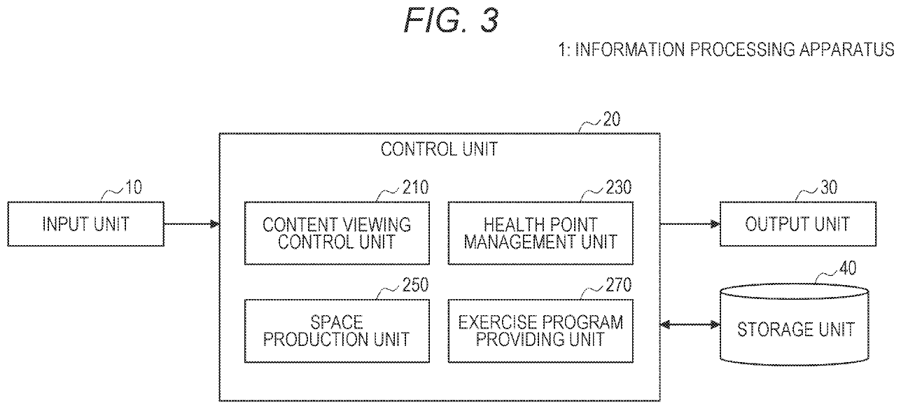

FIG. 3 is a block diagram illustrating an example of a configuration of the information processing apparatus 1 according to the present embodiment. As illustrated in FIG. 3, the information processing apparatus 1 includes an input unit 10, a control unit 20, an output unit 30, and a storage unit 40. Note that the information processing apparatus 1 may be realized by a large display device such as the television receiver (display unit 30a) as described with reference to FIG. 1, or may be realized by a portable television device, a personal computer (PC), a smartphone, a tablet terminal, a smart display, a projector, a game machine, or the like.

### Input Unit 10

The input unit 10 has a function of acquiring various types of information from the outside and inputting the acquired information to the information processing apparatus 1. More specifically, the input unit 10 may be, for example, a communication unit, an operation input unit, and a sensor.

The communication unit is communicably connected to an external device in a wired or wireless manner to transmit and receive data. For example, the communication unit is connected to a network and transmits and receives data to and from a server on the network. Furthermore, the communication unit may be communicably connected to an external device or a network by, for example, a wired/wireless local area network (LAN), Wi-Fi (registered trademark), Bluetooth (registered trademark), a mobile communication network (long term evolution (LTE), fourth generation mobile communication system (4G), and fifth generation mobile communication system (5G)), or the like. The communication unit according to the present embodiment receives, for example, a moving image distributed via a network. Furthermore, various output devices arranged in a space in which the information processing apparatus 1 is arranged are also assumed as the external device. Furthermore, a remote controller operated by a user is also assumed as the external device. The communication unit receives, for example, an infrared signal transmitted from a remote controller. Furthermore, the communication unit may receive a signal of television broadcasting (analog broadcasting or digital broadcasting) transmitted from the broadcasting station.

The operation input unit detects an operation by the user and inputs operation input information to the control unit 20. The operation input unit is realized by, for example, a button, a switch, a touch panel, or the like. Furthermore, the operation input unit may be realized by the above-described remote controller.

The sensor detects information of one or more users existing in the space, and inputs a detection result (sensing data) to the control unit 20. There may be a plurality of the sensors. In the present embodiment, the camera 10a is used as an example of the sensor. The camera 10a can acquire an RGB image as a captured image. The camera 10a may be a depth camera that can also acquire vibration information.

### Control Unit 20

The control unit 20 functions as an arithmetic processing device and a control device, and controls the overall operation in the information processing apparatus 1 according to various programs. The control unit 20 is realized by, for example, an electronic circuit such as a central processing unit (CPU) or a microprocessor. Furthermore, the control unit 20 may include a read only memory (ROM) that stores programs, operation parameters, and the like to be used, and a random access memory (RAM) that temporarily stores parameters and the like that change appropriately.

The control unit 20 according to the present embodiment also functions as a content viewing control unit 210, a health point management unit 230, a space production unit 250, and an exercise program providing unit 270.

The content viewing control unit 210 performs viewing control of various types of content in the content viewing mode M1. Specifically, control is performed to output video and audio of content distributed by a television program, a recorded program, or a moving image distribution service from the output unit 30 (display unit 30a, speaker 30b). The transition to the content viewing mode M1 can be performed by the control unit 20 according to a user operation.

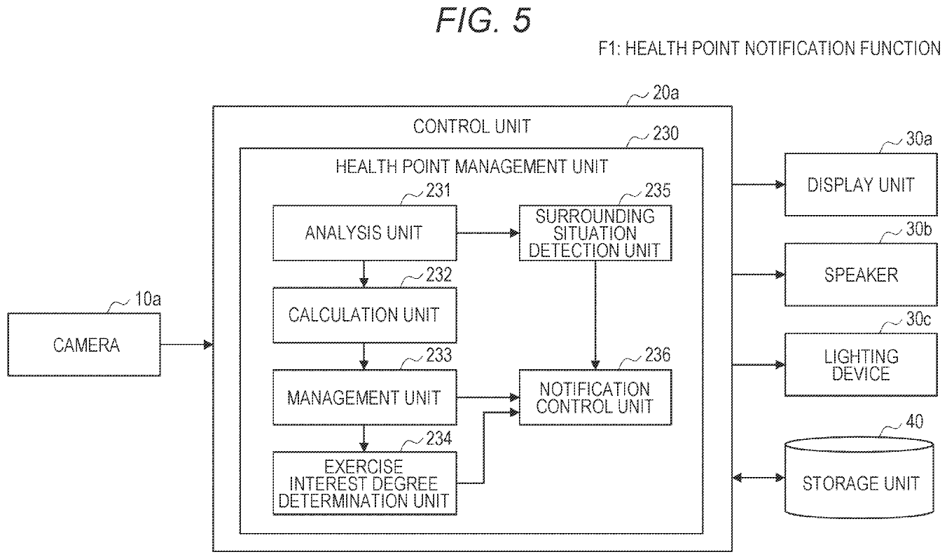

The health point management unit 230 realizes a health point notification function F1 that calculates and notifies the health point of the user. The health point management unit 230 can be implemented in both the content viewing mode M1 and the Well-being mode M2. The health point management unit 230 detects a healthful behavior from the user's behavior on the basis of the captured image acquired by the camera 10a included in the input unit 10 (further, using depth information), calculates corresponding health points, and grants the health points to the user. The granting to the user includes storing in association with the user information. Information on “healthy behavior” can be stored in advance in the storage unit 40. Furthermore, the information of the “healthful behavior” may be appropriately acquired from an external device. Furthermore, the health point management unit 230 notifies the user of information regarding the health points, such as the fact that the health points have been granted and the sum of the health points in a certain period. The notification to the user may be performed by the display unit 30a, or may be given to a personal terminal such as a smartphone or a wearable device possessed by the user. Details will be described later with reference to FIGS. 5 to 10.

The space production unit 250 determines the context of the user and realizes the space production function F2 for controlling the video, audio, and lighting for space production according to the context. The space production unit 250 can be implemented in the Well-being mode M2. The space production unit 250 performs control to output information for space production from, for example, display unit 30a, speaker 30b, and lighting device 30c installed in the space. The information for space production can be stored in advance in the storage unit 40. Furthermore, the information for space production may be acquired from an external device as appropriate. The transition to the Well-being mode M2 may be performed by the control unit 20 according to a user operation, or may be automatically performed by the control unit 20 determining the context.

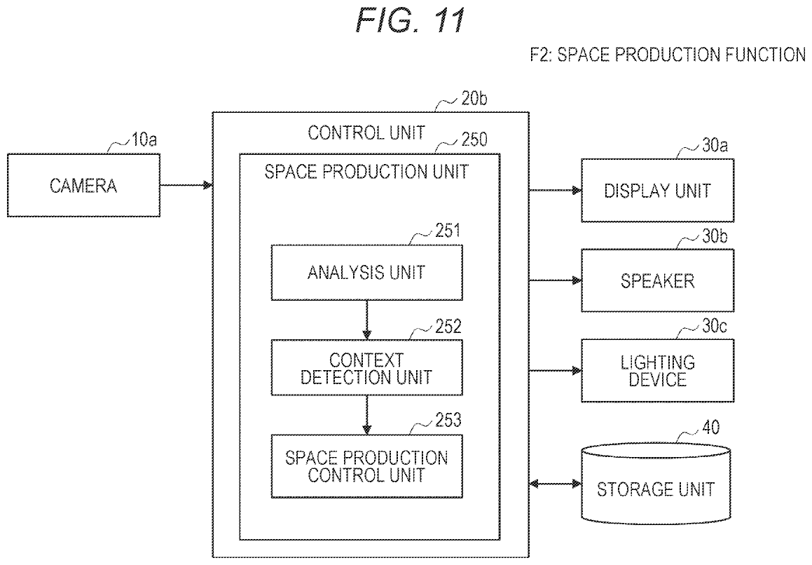

Details will be described later with reference to FIGS. 11 to 16.

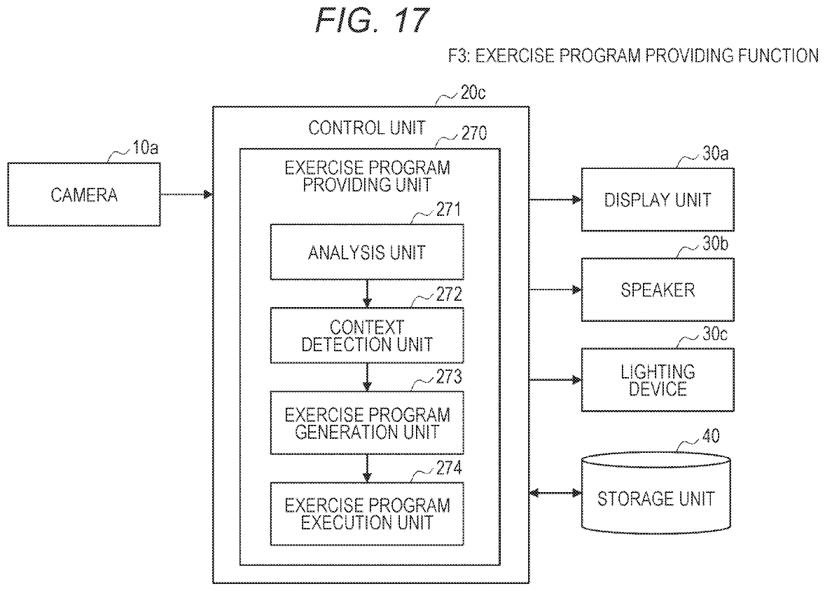

The exercise program providing unit 270 determines the context of the user, and realizes the exercise program providing function F3 that generates and provides an exercise program according to the context. The exercise program providing unit 270 can be implemented in the Well-being mode M2. The exercise program providing unit 270 provides the generated exercise program using, for example, the display unit 30a and the speaker 30b installed in the space. The information used to generate the exercise program and the generation algorithm can be stored in the storage unit 40 in advance. Furthermore, the information used for generating the exercise program and the generation algorithm may be appropriately acquired from an external device. Details will be described later with reference to FIGS. 17 to 21.

### Output Unit 30

The output unit 30 has a function of outputting various types of information under the control of the control unit 20. More specifically, the output unit 30 may be, for example, a display unit 30a, a speaker 30b, and a lighting device 30c. The display unit 30a may be realized by, for example, a large display device such as a television receiver, or may be realized by a portable television device, a personal computer (PC), a smartphone, a tablet terminal, a smart display, a projector, a game machine, or the like.

### Storage Unit 40

The storage unit 40 is realized by a read only memory (ROM) that stores programs, operation parameters, and the like used for processing of the control unit 20, and a random access memory (RAM) that temporarily stores parameters and the like that change appropriately. For example, the storage unit 40 stores information on a healthy behavior, an algorithm for calculating health points, various types of information for space production, information for generating an exercise program, an algorithm for generating an exercise program, and the like.

Although the configuration of the information processing apparatus 1 has been specifically described above, the configuration of the information processing apparatus 1 according to the present disclosure is not limited to the example illustrated in FIG. 3. For example, the information processing apparatus 1 may be implemented by a plurality of devices. Specifically, for example, the system may include a display device including the display unit 30a, the control unit 20, the communication unit, and the storage unit 40, the speaker 30b, and the lighting device 30c. Furthermore, the control unit 20 may be realized by a device separate from the display unit 30a. Furthermore, at least a part of the function of the control unit 20 may be realized by an external control device. As the external control device, for example, a PC, a tablet terminal, a smartphone, or a server (cloud server, edge server, etc.) is assumed. Furthermore, at least a part of each piece of information stored in the storage unit 40 may be stored in an external storage device or server (cloud server, edge server, etc.).

Furthermore, the sensor is not limited to the camera 10a. For example, a microphone, an infrared sensor, a thermo sensor, an ultrasonic sensor, or the like may be further included. Furthermore, the speaker 30b is not limited to the mounting type illustrated in FIG. 1. The speaker 30b may be realized by, for example, a headphone, an earphone, a neck speaker, a bone conduction speaker, or the like. Furthermore, a plurality of the speakers 30b may be provided. Furthermore, in a case where there is a plurality of the speakers 30b communicatively connected to the control unit 20, the user may arbitrarily select from which speaker 30b the voice is output.

### 3. Operation Processing

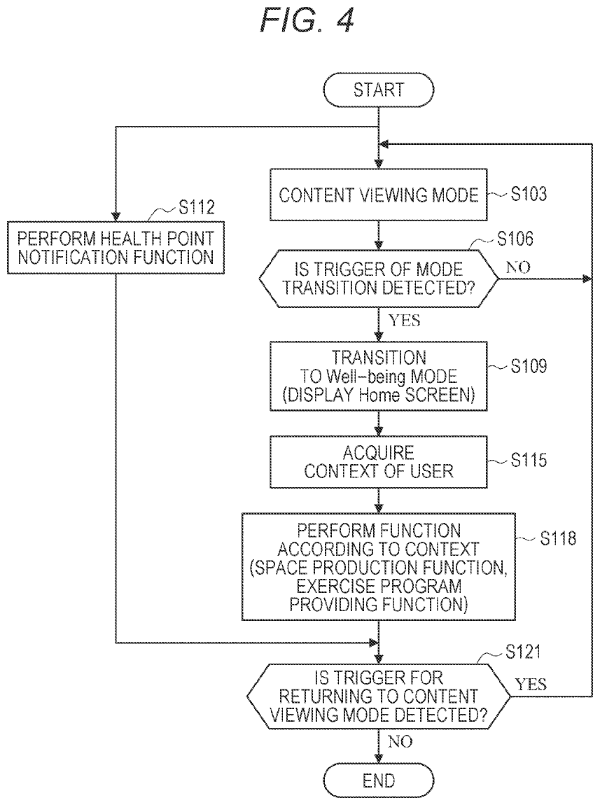

FIG. 4 is a flowchart illustrating an example of a flow of entire operation processing for implementing various functions according to the present embodiment.

As illustrated in FIG. 4, first, in the content viewing mode, the content viewing control unit 210 of the control unit 20 performs control to output content (video image and audio) appropriately designated by the user from the display unit 30a or the speaker 30b (step S103).

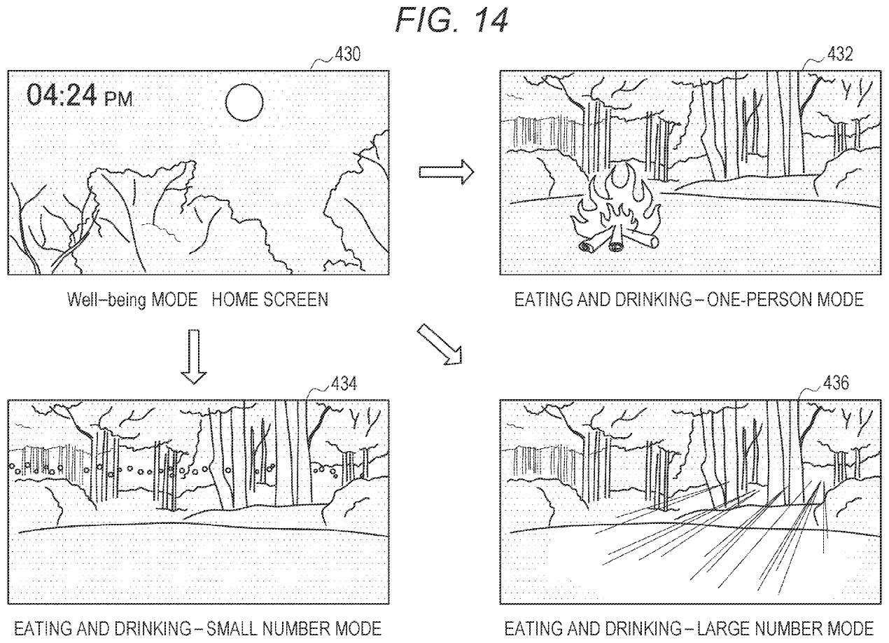

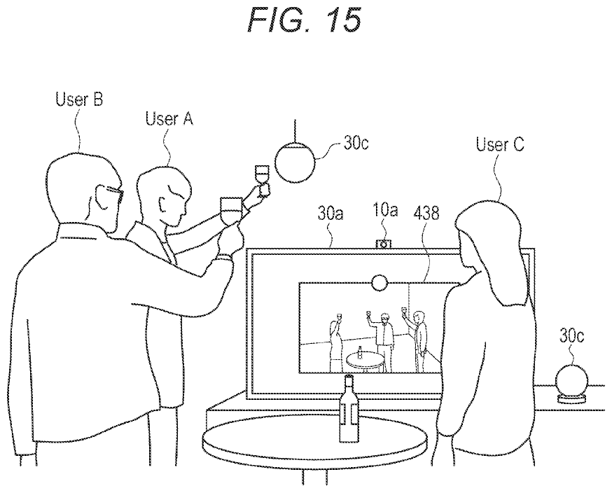

Next, in a case where the trigger of the mode transition is detected (step S106/Yes), the control unit 20 performs control to transition the operation mode of the information processing apparatus 1 to the Well-being mode. A trigger of the mode transition may be an explicit operation by the user or may be a case where a predetermined context is detected. The predetermined context is, for example, that the user is not looking at the display unit 30a, is doing something other than content viewing, or the like. The control unit 20 can analyze the posture and movement, biometric information, face orientation, and the like of one or more users (persons) existing in the space from the captured image continuously acquired by the camera 10a, and determine the context. The control unit 20 displays a predetermined home screen immediately after transitioning to the Well-being mode. Although FIG. 14 illustrates a specific example of the home screen, the home screen may be an image of a natural landscape or a static landscape, for example. The image of the home screen is desirably a video that does not disturb the user who is doing something other than content viewing.

On the other hand, the control unit 20 continuously performs the health point notification function F1 during the content viewing mode or when transitioning to the Well-being mode (step S112). Specifically, the health point management unit 230 of the control unit 20 analyzes a posture, movement, and the like of one or more users (persons) existing in a space from the captured image continuously acquired by the camera 10a, and determines whether or not a healthy behavior (posture, movement, etc.) is performed. In a case where a healthy behavior is performed, the health point management unit 230 grants health points to the user. Note that, by registering the face information of each user in advance, the health point management unit 230 can identify the user by face analysis from the captured image and store the health points in association with the user. Furthermore, the health point management unit 230 performs control to notify the user of the granting of the health points from the display unit 30a or the like at a predetermined timing. The notification to the user may be displayed on the home screen displayed immediately after the transition to the Well-being mode.

Next, after transitioning to the Well-being mode, the control unit 20 analyzes the captured image acquired from the camera 10a and acquires the context of the user (step S115). Note that the context may be continuously acquired from the content viewing mode. In the analysis of the captured image, for example, face recognition, object detection, action (motion) detection, posture estimation, and the like can be performed.

Next, the control unit 20 performs a function according to the context among various functions (applications) provided in the Well-being mode (step S118). In the present embodiment, functions that can be provided according to the context include the space production function F2 and the exercise program providing function F3. The application (program) for executing each function may be stored in the storage unit 40 in advance, or may be acquired from a server on the Internet as appropriate. In a case where the context defined by each function is detected, the control unit 20 implements the corresponding function. The context is a surrounding situation, and includes, for example, at least one of the number of users, an object held in a hand of the user, things being performed/to be performed by the user, a state of biometric information (pulse, body temperature, facial expression, etc.), an excitement degree (voice size, voice amount, handling given, etc.), or a gesture.

Furthermore, the health point management unit 230 of the control unit 20 can continuously implement the health point notification function F1 even during the Well-being mode. For example, even while the space production function F2 is performed, the health point management unit 230 detects a healthy behavior from the posture and movement of each user and grants health points as appropriate. The notification of the health points may be turned off while the space production function F2 is performed so as not to disturb the space production. Furthermore, for example, the health point management unit 230 grants the health points according to the exercise program (exercise performed by the user) provided by the exercise program providing function F3. The notification of the health points may be performed at the time when the exercise program ends.

Then, in a case where a trigger for returning to the content viewing mode is detected (step S121/Yes), the control unit 20 causes the operation mode to transition from the Well-being mode to the content viewing mode (step S103). The mode transition trigger may be an explicit operation by the user.

The entire operation processing according to the present embodiment has been described above. Note that the above-described operation processing is an example, and the present disclosure is not limited thereto.

Furthermore, the explicit operation by the user in triggering the mode transition may be a voice input by the user. Furthermore, the specification of the user is not limited to the face recognition based on the captured image, and may be voice authentication based on a user's utterance voice collected by a microphone that is an example of the input unit 10. Furthermore, the acquisition of the context is not limited to the analysis of the captured image, and analysis of the utterance voice or the environmental sound collected by the microphone may be further used.

Hereinafter, each of the above-described functions will be specifically described with reference to the drawings.

### 4. First Example (Health Point Notification Function)

As a first example, the health point notification function will be specifically described with reference to FIGS. 5 to 10.

### 4-1. Configuration Example

FIG. 5 is a block diagram illustrating an example of a configuration of the information processing apparatus 1 that implements the health point notification function according to the first example. As illustrated in FIG. 5, the information processing apparatus 1 that implements the health point notification function includes a camera 10a, a control unit 20a, a display unit 30a, a speaker 30b, a lighting device 30c, and a storage unit 40. The camera 10a, the display unit 30a, the speaker 30b, the lighting device 30c, and the storage unit 40 are as described with reference to FIG. 3, and thus detailed description thereof is omitted here.

The control unit 20a functions as a health point management unit 230. The health point management unit 230 has functions of an analysis unit 231, a calculation unit 232, a management unit 233, an exercise interest degree determination unit 234, a surrounding situation detection unit 235, and a notification control unit 236.

The analysis unit 231 analyzes the captured image acquired by the camera 10a, and detects skeleton information and face information. In the detection of the face information, it is possible to specify the user by comparing the face information with the face information of each user registered in advance. The face information is, for example, information of feature points of the face. The analysis unit 231 compares the feature points of the face of the person analyzed from the captured image with the feature points of the face of one or more users registered in advance, and specifies a user having a matching feature (face recognition processing). Furthermore, in the detection of the skeleton information, for example, each part (head, shoulder, hand, foot, and the like) of each person is recognized from the captured image, and the coordinate position of each part is calculated (acquisition of joint position). Furthermore, the detection of the skeleton information may be performed as posture estimation processing.

Next, the calculation unit 232 calculates health points on the basis of the analysis result output from the analysis unit 231. Specifically, the calculation unit 232 determines whether or not the user has performed a pre-registered “healthful behavior” on the basis of the detected skeleton information of the user, and calculates a corresponding health point in a case where the user has performed the “healthful behavior”. The “healthy behavior” is a predetermined posture or movement. For example, the action may be a stretch item such as “stretch” in which both arms are raised above the head, a healthy action (walk, laugh) often seen in the living room, or the like. Furthermore, muscle strength training, exercise, dancing, housework, and the like are also included. The storage unit 40 may store a list of “healthful behaviors”.

In each item in the list, a name of “healthy behavior”, skeleton information, and a difficulty level are associated. The skeleton information may be the point group information itself of the skeleton obtained by the skeleton detection, or may be information such as a characteristic angle formed by two or more line segments connecting points of the skeleton with lines. The difficulty level may be predetermined by an expert. In the case of stretching, the difficulty level can be determined from the difficulty of the pose. Furthermore, the difficulty level may be determined by the magnitude of the motion of the body from the normal posture (sitting posture, standing posture) to the pause (In a case where the motion is large, the difficulty level is high, and in a case where the motion is small, the difficulty level is low.). Furthermore, in the case of muscle strength training, exercise, or the like, the higher the load on the body, the higher the difficulty level may be determined.

The calculation unit 232 may calculate the health points according to the difficulty level of “healthy behavior” matching the posture or movement performed by the user. For example, the calculation unit 232 calculates the difficulty level and the health points on the basis of a database in which the difficulty level and the health points are associated with each other. Furthermore, the calculation unit 232 may calculate the health point by giving a weight according to the difficulty level to the basis point for performing the “healthful behavior”. Furthermore, the calculation unit 232 may vary the difficulty level according to the ability of the user. The ability of the user can be determined on the basis of the accumulation of the behavior of the user. The ability of the user may be divided into three stages of “beginner, intermediate person, and advanced person”. For example, the difficulty level of a certain stretch item included in the list may be generally “medium”, but may be changed to “high” in a case of being applied to a beginner user. Note that the “difficulty level” can also be used when the user is recommended to stretch or the like.

Furthermore, after calculating the health points for a certain healthy behavior, the calculation unit 232 may not calculate the health points for the same behavior within a predetermined time (for example, 1 hour), or may calculate the health points by reducing the health points by a predetermined ratio. Furthermore, the calculation unit 232 may add bonus points in a case where a preset number of healthy behaviors are detected in one day.

The management unit 233 stores the health points calculated by the calculation unit 232 in the storage unit 40 in association with the information of the user. In the storage unit 40, identification information (facial feature point or the like), a user name, a height, a weight, skeleton information, a hobby, and the like can be stored in advance as information of one or more users. The management unit 233 stores information regarding the health points granted to the corresponding user as one of the information regarding the user. The information regarding the health points includes a detected behavior (a name or the like extracted from the list item), health points granted to the user according to the behavior, a date and time when the health points are granted, and the like.

The health points described above may be used to add materials of various applications. Furthermore, it may be used as a point for opening a new application in the Well-being mode or opening a function of each application in the Well-being mode. Furthermore, it may be used for product purchase.

The exercise interest degree determination unit 234 determines an interest degree of the user in exercise on the basis of the health points. Since the health points of each user are accumulated, the exercise interest degree determination unit 234 may determine the interest degree of the user in exercise on the basis of the sum of the health points for a certain period (for example, one week). For example, it can be determined that the higher the health points, the higher the degree of interest in exercise. More specifically, for example, the exercise interest degree determination unit 234 may determine the degree of interest in exercise as follows according to the total of the health points for one week.
0 P . . . No interest in exercise (Level 1)
0 to 100 P . . . Somewhat interested in exercise (Level 2)
100 to 300 P . . . Interest in exercise (Level 3)
300 P . . . Very interested in exercise (Level 4)

A threshold of the points at each level may be determined according to the score of each behavior registered in the list and verification of how much score can be generally acquired in a certain period.

Furthermore, the exercise interest degree determination unit 234 may make the determination not by a predetermined level (absolute evaluation) but by comparison with the past state of the user (relative evaluation). For example, the exercise interest degree determination unit 234 determines that “the user has become very interested in exercise” if the user's total health points for each week have increased by a predetermined point (for example, 100 P) or more from last week due to a change (temporal change) in the total health points of the user. Furthermore, the exercise interest degree determination unit 234 determines that “the interest in exercise is weakened” if the total of the health points has decreased by a predetermined point (for example, 100 P) or more from last week. Furthermore, the exercise interest degree determination unit 234 determines that “the interest in the exercise is stable” if a difference from the previous week is less than or equal to a predetermined point (for example, 50 P). A width of the score may also be determined by verification.

The surrounding situation detection unit 235 detects the surrounding situation (so-called context) on the basis of the analysis result of the captured image by the analysis unit 231. For example, the surrounding situation detection unit 235 detects whether there is a user who is looking at the display unit 30a, whether there is a user who is concentrating on the content being reproduced on the display unit 30a, or whether there is a user who is in front of the display unit 30a but not concentrating on the content (not looking, doing other things). Whether or not the user is looking at the display unit 30a can be determined from a face direction and a body direction (posture) of each user obtained from the analysis unit 231. Furthermore, in a case where the user keeps looking at the display unit 30a for a predetermined time or more, it can be determined that the user is concentrating. Furthermore, in a case where eye blinks, line-of-sight, and the like are also detected as the face information, it is also possible to determine the degree of concentration on the basis of these.

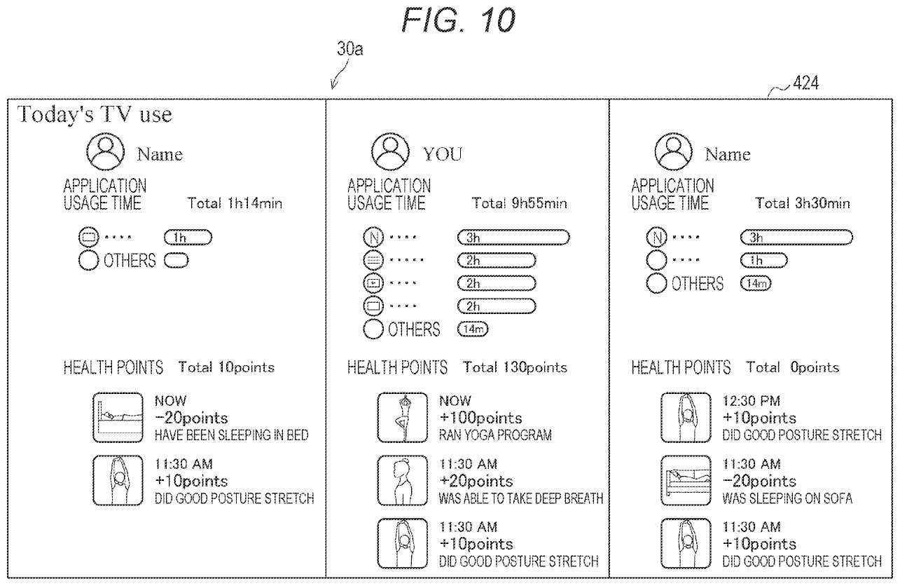

The notification control unit 236 performs control to notify the user of information regarding the health points granted to the user by the management unit 233 at a predetermined timing. The notification control unit 236 may perform the notification at timing when the context detected by the surrounding situation detection unit 235 satisfies the condition. For example, since notifying the display unit 30a in a case where there is a user who concentrates on content hinders content viewing, the notification may be made from the display unit 30a in a case where the user does not concentrate on the content, in a case where the user is not looking at the display unit 30a, or in a case where the user is doing something other than content viewing. The notification control unit 236 may determine whether or not the context satisfies the condition when the health points are granted by the management unit 233. In a case where the context does not satisfy the condition, the notification may be performed after waiting until a satisfaction timing. Furthermore, the display of the information regarding the health points may be performed in response to an explicit operation by the user (confirmation of the health points. See FIG. 10.).

Furthermore, the notification control unit 236 may determine the contents of the notification according to the interest degree of the user in exercise determined by the exercise interest degree determination unit 234. The contents of the notification include, for example, health points to be granted this time, a reason for the granting, an effect brought by the behavior, a recommended stretch, and the like, and a timing of making a recommendation.

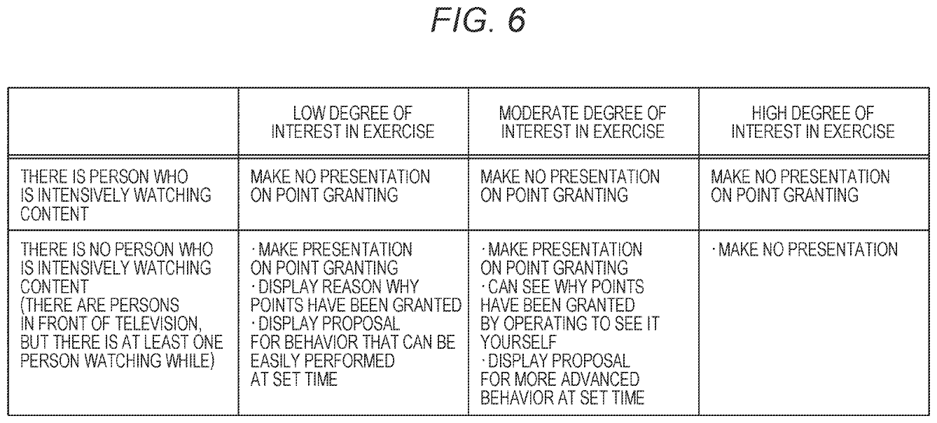

Here, FIG. 6 illustrates an example of notification contents according to the degree of interest in exercise according to the first example. As illustrated in FIG. 6, in a case where there is a person who is intensively watching content, the notification control unit 236 does not present information regarding point granting in any case. On the other hand, in a case where there is no person who is intensively watching content, the notification control unit 236 determines the notification contents as shown in the table according to the interest degree of the user in exercise.

For example, the user having a low degree of interest in exercise is notified of the fact that the health points have been granted, the reason for the granting, and the like. These pieces of information may be simultaneously displayed on the screen of the display unit 30a, or may be sequentially displayed. Furthermore, the display unit 30a notifies a user having a low degree of interest in exercise of a proposal for a “healthful behavior” (stretch or the like) that can be easily performed at a time determined by the system side (for example, 21:00 which is a night leisure time) or at a time determined by the user and in a case where there is no person who is intensively watching content. The term “easily performed” is assumed to be a stretch with a low difficulty level, a stretch without using a tool such as a chair or a towel, or the like. Furthermore, a stretch or the like that can be performed without changing the posture from the current posture of the user is assumed. That is, a stretch or the like with a low psychological hurdle (motivation occurs) for the user with a low degree of interest in exercise is proposed.

Furthermore, in the case of a person having a moderate degree of interest in exercise, notification only that health points have been granted is given. The reason for the granting may be displayed in accordance with a user operation.

Furthermore, the display unit 30a notifies a user having a moderate degree of interest in exercise of a proposal for a more advanced “healthy behavior” (stretch or the like) at a time determined by the system side or at a time determined by the user and in a case where there is no person who is intensively watching content. The term “advanced” is assumed to be a stretch with high difficulty, a stretch using a tool such as a chair or a towel, or the like. Furthermore, a stretch or the like performed by greatly changing a posture from the current posture of the user is assumed. This is because there is a high possibility that the user having a moderate degree of interest in exercise performs the stretching or the like even if the stretching or the like has a high psychological hurdle.

Note that how to select the recommended stretch or the like for the user is not limited to the difficulty level. For example, the notification control unit 236 may grasp the user's usual posture or the tendency of movement in the room in one day, and propose an appropriate stretch or the like. Specifically, in a case where the user is sitting all the time or a person who does not move his/her body on a daily basis, the recommendation may be sequentially presented by a configuration of stretching to stretch the muscles of the entire body such that the next recommendation is displayed when the user can perform one recommended stretch. Furthermore, in a case where the motion is constant during the day, a recommended behavior (for example, deep breathing, yoga pose, and the like) having a configuration for creating a relaxed state may be presented. Furthermore, it is also possible to adopt a configuration in which the user does not damage a body part when presenting a recommended stretch or the like by storing pain information or the like of the body part in advance.

Note that, in a case where the person has a high degree of interest in exercise, no presentation may be performed. Since there is a high possibility that a person who is highly interested in exercise performs stretching or the like in a spare time or creates time to move the body without making a proposal from the system side, the person does not make any notification, thereby reducing botheration caused by the notification.

Furthermore, in a case where the home screen in the Well-being mode is displayed, since the user is not viewing the content, the notification control unit 236 may determine that “there is no person who is intensively watching content” and perform the notification.

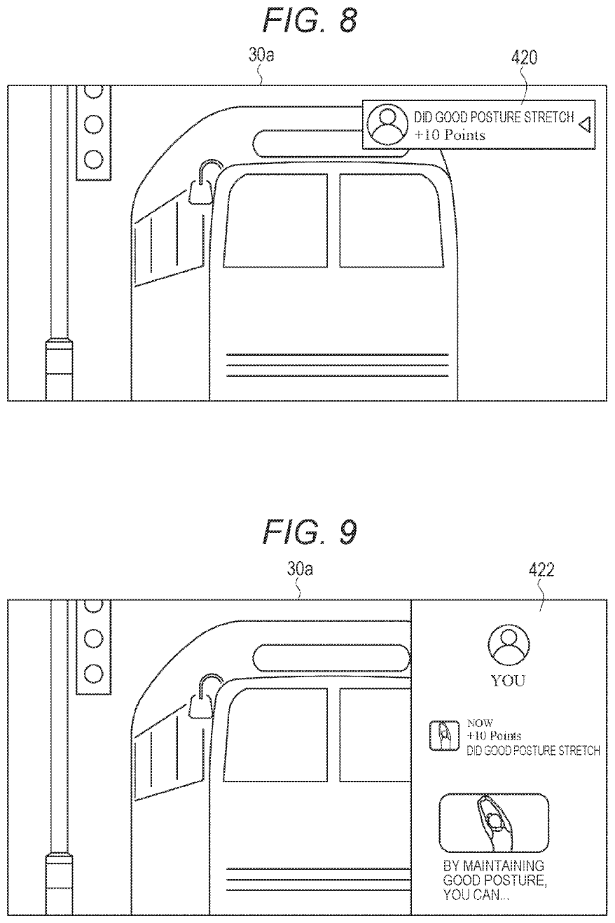

Furthermore, the manner of notification by the notification control unit 236 may be such that the notification image fades in on the screen of the display unit 30a and is displayed for a certain period of time and then fades out, or the notification image may slide in on the screen of the display unit 30a, is displayed for a certain period of time, and then slides out (See FIGS. 8 and 9).

Furthermore, the notification control unit 236 may also perform control of audio and lighting at the time of performing notification by display.

The configuration for realizing the health point notification function according to the present example has been specifically described above. Note that the configuration according to the present example is not limited to the example illustrated in FIG. 5. For example, the configuration for realizing the health point notification function may be realized by one device or may be realized by a plurality of devices. Furthermore, the control unit 20a, the camera 10a, the display unit 30a, the speaker 30b, and the lighting device 30c may be communicably connected to each other in a wireless or wired manner. Furthermore, at least one of the display unit 30a, the speaker 30b, or the lighting device 30c may be included. Furthermore, a configuration further including a microphone may be employed.

Furthermore, in the description described above, it has been described that the health points are granted by detecting the “healthy behavior”, but the present example is not limited thereto. For example, “unhealthy behavior” may also be detected, and the health point may be deducted. Information regarding “unhealthy behavior” can be registered in advance. Examples thereof include bad posture, keep sitting, and sleeping on a sofa.

### 4-2. Operation Processing

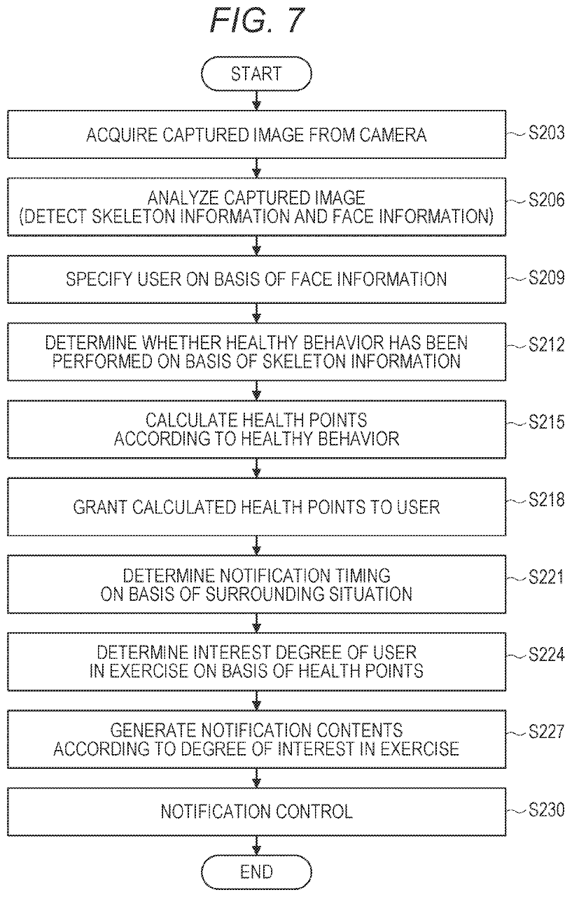

Next, operation processing according to the present example will be described with reference to FIG. 7. FIG. 7 is a flowchart illustrating an example of a flow of a health point notification process according to the first example.

As illustrated in FIG. 7, first, a captured image is acquired by the camera 10a (step S203), and the analysis unit 231 analyzes the captured image (step S206). In the analysis of the captured image, for example, skeleton information and face information are detected.

Next, the analysis unit 231 specifies the user on the basis of the detected face information (step S209).

Next, the calculation unit 232 determines whether the user has performed a healthy behavior (good posture, stretch, etc.) on the basis of the detected skeleton information (step S212), and calculates health points according to the healthy behavior performed by the user (step S215).

Subsequently, the management unit 233 grants the calculated health points to the user (step S218). Specifically, the management unit 233 stores the calculated health points in the storage unit 40 as information of the specified user.

Next, the notification control unit 236 determines the notification timing on the basis of the surrounding situation (context) detected by the surrounding situation detection unit 235 (step S221). Specifically, the notification control unit 236 determines whether or not the context satisfies a predetermined condition that a notification may be made (for example, there is no person who is intensively watching the content).

Next, the exercise interest degree determination unit 234 determines the interest degree of the user in exercise according to the health points (step S224).

Then, the notification control unit 236 generates notification contents according to the interest degree of the user in the exercise (step S227), and notifies the user of the notification contents (step S230). Here, FIGS. 8 and 9 illustrate examples of the health point notification to the user according to the first example.

As illustrated in FIG. 8, for example, the notification control unit 236 may display, on the display unit 30a, an image 420 indicating that the health points have been granted to the user and the reason for the granting by fade-in, fade-out, pop-up, or the like for a certain period of time. Furthermore, as illustrated in FIG. 9, for example, the notification control unit 236 may display, on the display unit 30a, an image 422 describing that the health points have been granted to the user, the reason for the granting, and the effect thereof by fade-in, fade-out, pop-up, or the like for a certain period of time.

Furthermore, the notification control unit 236 may display a health point confirmation screen 424 as illustrated in FIG. 10 on the display unit 30a in response to an explicit operation by the user. On the confirmation screen 424, the total of the daily health points of each user and the breakdown thereof are displayed. Furthermore, on the confirmation screen 424, a content viewing time or the like (For example, how many hours the user watched TV, how many hours the user played a game, and how many hours the user used which video distribution service.) by each service may be displayed together. In addition to the explicit operation by the user, the confirmation screen 424 may be displayed for a certain period of time when transitioning to the Well-being mode, may be displayed for a certain period of time when the power of the display unit 30a is turned off, or may be displayed for a certain period of time before sleeping time.

The operation processing of the health point notification function according to the present example has been described above. Note that the flow of the operation processing illustrated in FIG. 7 is an example, and the present example is not limited thereto. For example, the order of the steps illustrated in FIG. 7 may be processed in parallel, may be processed in reverse, or may be skipped.

### 4-3. Modified Example

Next, a modified example of the first example will be described.

In the above-described example, it has been described that the user is specified on the basis of the face information, but the present disclosure is not limited thereto, and the analysis unit 231 may use, for example, object information. The object information is obtained by analyzing the captured image. More specifically, the analysis unit 231 may specify the user by the color of the clothes worn by the user. When the user can be specified in advance by face recognition, the management unit 233 newly registers the color of the clothes worn by the user (as the information of the user in the storage unit 40). As a result, even in a case where the face recognition cannot be performed, the color of the clothes worn by the person can be determined from the object information obtained by analyzing the captured image, and the user can be specified. For example, even in a case where the user's face is not shown (for example, in a case where the user extends backward with respect to the camera,), the user can be identified, and the health points can be granted. Note that the analysis unit 231 can also specify the user from other data other than the object information. For example, the analysis unit 231 identifies who is where on the basis of a communication result with a smartphone, a wearable device, or the like possessed by the user, and identifies a person shown by merging with skeleton information or the like acquired from the captured image. For the position detection by communication, for example, a position detection technology by Wi-Fi is used.

Furthermore, in a case where a healthy behavior is detected but the user cannot be specified, the management unit 233 may not grant the health points to anyone or may grant the health points at a predetermined ratio to all family members.

Furthermore, in the above-described example, a case where there is no user who is intensively viewing the content has been described as an example of the notification control according to the context, but the present example is not limited thereto. For example, the object recognition is performed from the captured image, the object held in the hand of the user is recognized, and in a case where the user holds a smartphone or a book, there is a possibility that stretching or the like is performed while concentrating on the smartphone or the book. Therefore, notification by sound may not be performed so as not to disturb concentration (notification is performed only on the screen). Furthermore, since there is a possibility that the speech voice collected by the microphone is analyzed and stretching or the like is performed while being absorbed in the conversation, notification by sound may not be performed (notification is performed only on the screen) so as not to disturb the conversation. In this manner, a more detailed context may be detected, and appropriate presentation may be performed according to the context.

Furthermore, as a notification method, notification on a screen, notification by sound (communication sound), and notification by illumination (lighting is brightened, changed to predetermined color, blinking etc.) may be performed at the same timing, or may be used properly according to a situation. For example, in a case where “there is a person who is intensively viewing the content”, notification is not performed in the above-described example, but notification other than screen and sound, for example, only notification by lighting may be performed. Furthermore, in a case where “there is no person who is intensively viewing content”, it can be determined that the user is viewing the screen from the face information, and in a case where it is determined that the user is standing from the skeleton information, the notification control unit 236 may perform notification on the screen and notification with lighting, and may turn off the notification with sound (communication sound) (since there is a high possibility that the user notices the notification on the screen without sounding the notification sound). On the other hand, in other cases, the notification control unit 236 may perform notification on the screen, notification by sound, and notification by lighting together. Furthermore, in a case where the atmosphere performance is performed in the Well-being mode, the notification control unit 236 may perform the notification only by the screen and the illumination without performing the notification by sound so as not to destroy the atmosphere, may perform the notification only by either the screen or the lighting, or may not perform the notification by any method.

Furthermore, as the notification timing, in a case where the user is viewing a specific content, the notification may not be performed (at least, the notification by screen and sound is not performed). For example, it is assumed that a genre (drama, movie, news, etc.) of content desired to be intensively viewed by the user is registered in advance. As a result, the notification control unit 236 may not perform notification with a screen or a sound in a case where the user intensively views content desired to be viewed, and may perform notification with a screen or a sound in a case where the user views content of other genres.

Furthermore, the “specific content” described above may be detected and registered on the basis of the user's usual habit. For example, the surrounding situation detection unit 235 integrates the user's face information and posture information with the genre of the content, and specifies the genre of the content that the user is watching for a relatively long time. More specifically, for example, the surrounding situation detection unit 235 measures the rate at which the user has viewed the screen for each genre in the time during which the user has viewed the content in one week (the time when the front face could be detected, the rate obtained by dividing the time when the face was directed to the television by the content broadcast time, and the like), and determines which genre of the content the user has frequently viewed the screen in. As a result, it is possible to register a genre (specific content) that the user is estimated to intensively want to view. The estimation of the genre may be updated every time the broadcast or distributed content is switched, or may be updated by measuring every month or every week.

### 5. Second Example (Space Production Function)

Next, as a second example, the space production function will be specifically described with reference to FIGS. 11 to 16. In the present example, according to the human context, it is possible to perform music and lighting that further enhance the concentration of the human, production of an atmosphere that promotes the physical and mental health state of the human, production of a relaxing environment, production that further enhances the state in which the human is enjoying, and the like.

In such performance, as an example, a natural landscape (forest, starry sky, lake, sea, waterfall, etc.) or a natural sound (sound of river, sound of wind, cry of insect, and the like) is used. In recent years, urbanization of various places has progressed, and it tends to be difficult to feel nature from a living space. Since there are few opportunities to come in contact with nature and stress is likely to be felt, natural elements are incorporated into the living space by creating a space like being in nature by sound and video, thereby reducing malaise, recovering energy, and improving productivity.

### 5-1. Configuration Example

FIG. 11 is a block diagram illustrating an example of a configuration of an information processing apparatus 1 that realizes a space production function according to the second example. As illustrated in FIG. 11, the information processing apparatus 1 that implements the space production function includes a camera 10a, a control unit 20b, a display unit 30a, a speaker 30b, a lighting device 30c, and a storage unit 40. The camera 10a, the display unit 30a, the speaker 30b, the lighting device 30c, and the storage unit 40 are as described with reference to FIG. 3, and thus detailed description thereof is omitted here.

The control unit 20b functions as the space production unit 250. The space production unit 250 has functions of an analysis unit 251, a context detection unit 252, and a space production control unit 253.

The analysis unit 251 analyzes the captured image acquired by the camera 10a, and detects skeleton information and object information. In the detection of the skeleton information, for example, each part (head, shoulder, hand, foot, and the like) of each person is recognized from the captured image, and the coordinate position of each part is calculated (acquisition of joint position). Furthermore, the detection of the skeleton information may be performed as posture estimation processing. Furthermore, in the detection of the object information, an object existing in the periphery is recognized. Furthermore, the analysis unit 251 can also integrate skeleton information and object information to recognize an object held in the hand of the user.

The context detection unit 252 detects the context on the basis of the analysis result of the analysis unit 251. More specifically, the context detection unit 252 detects the situation of the user as the context. Examples thereof include eating and drinking, talking with several people, doing housework, relaxing alone, reading a book, falling asleep, getting up, and preparing for going out. These are examples, and various situations may be detected. Note that the algorithm for context detection is not particularly limited. The context detection unit 252 may detect the context with reference to information such as an assumed posture, a place where the user is, and belongings in advance.

The space production control unit 253 performs control to output various kinds of information for space production according to the context detected by the context detection unit 252. Various types of information for space production according to the context may be stored in advance in the storage unit 40, may be acquired from a server on the network, or may be newly generated. In the case of newly generating, generation may be performed according to a predetermined generation algorithm, generation may be performed by combining predetermined patterns, or generation may be performed using machine learning. Examples of the various types of information include video, audio, and lighting patterns. As described above, a natural landscape and a natural sound are assumed as an example. Furthermore, the space production control unit 253 may select and generate various kinds of information for space production according to the context and the preference of the user. By outputting various types of information for space production according to the context, it is possible to perform presentation or the like to further enhance the concentration of a person, promote the health state of the human body and mind, present a relaxing environment, or further enhance the state in which the person enjoys.

The configuration for realizing the space production function according to the present example has been specifically described above. Note that the configuration according to the present example is not limited to the example illustrated in FIG. 11. For example, the configuration for realizing the space production function may be realized by one device or may be realized by a plurality of devices. Furthermore, the control unit 20b, the camera 10a, the display unit 30a, the speaker 30b, and the lighting device 30c may be communicably connected to each other in a wireless or wired manner. Furthermore, at least one of the display unit 30a, the speaker 30b, or the lighting device 30c may be included. Furthermore, a configuration further including a microphone may be employed.

### 5-2. Operation Processing

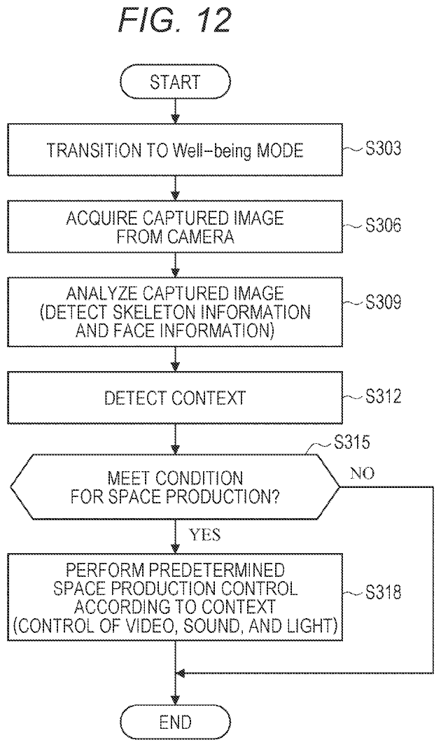

Next, operation processing according to the present example will be described with reference to FIG. 12. FIG. 12 is a flowchart illustrating an example of a flow of space production processing according to the second example.

As illustrated in FIG. 12, first, the control unit 20b transitions the operation mode of the information processing apparatus 1 from the content viewing mode to the Well-being mode (step S303). The transition to the Well-being mode is as described in step S106 of FIG. 4.

Next, a captured image is acquired by the camera 10a (step S306), and the analysis unit 251 analyzes the captured image (step S309). In the analysis of the captured image, for example, skeleton information and object information are detected.

Next, the context detection unit 252 detects the context on the basis of the analysis result (step S312).

Next, the space production control unit 253 determines whether or not the detected context meets a preset condition for space production (step S315).

Next, in a case where the detected context meets the condition (step S315/Yes), the space production control unit 253 performs predetermined space production control according to the context (step S318). Specifically, for example, control (control of video, sound, and light) for outputting various types of information for space production according to the context is performed. Note that, here, as an example, a case where the predetermined condition is satisfied has been described. However, the present example is not limited thereto, and in a case where the information for space production corresponding to the detected context is not prepared in the storage unit 40, the space production control unit 253 may newly acquire the information from the server, or the space production control unit 253 may newly generate the information.

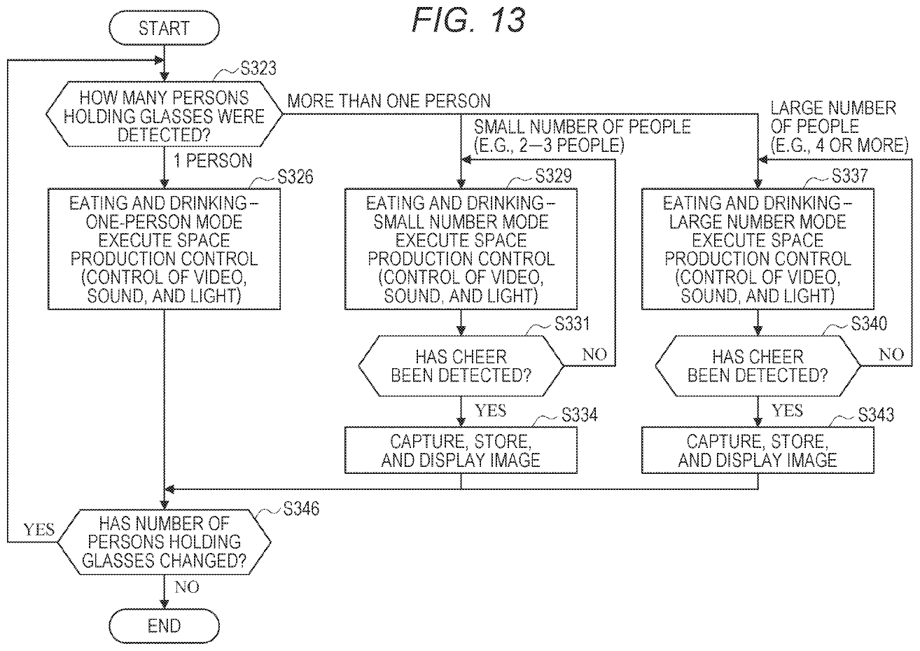

The flow of the space production processing according to the present example has been described above. Note that the space production control shown in step S318 described above will be further specifically described with reference to FIG. 13. In FIG. 13, as a specific example, space production control in a case where the context is “eating and drinking” will be described.

FIG. 13 is a flowchart illustrating an example of a flow of space production processing during eating and drinking according to the second example. This flow is performed in a case where the context is “eating and drinking”.

As illustrated in FIG. 13, first, the space production control unit 253 performs space production control according to the number of persons (more specifically, for example, the number of persons holding glasses (drinks)) who are eating and drinking, indicated by the detected context (steps S323, S326, S329, and S337). A person who is eating and drinking, each person holding a glass, and the like can be detected on the basis of skeleton information (posture, hand shape, arm shape, and the like) and object information. For example, in a case where the glass is detected by object detection, and further, it is found that the position of the glass and the position of the wrist are within a certain distance from the object information and the skeleton information, it can be determined that the user has the glass. Once the object is detected, it may be estimated that the user is holding the object for a certain period of time thereafter while the user is not moving. Furthermore, in a case where the user has moved, object detection may be newly performed.

Here, an example of space production according to the number of people eating and drinking is illustrated in FIG. 14. FIG. 14 is a diagram illustrating an example of a video for space production according to the number of people eating and drinking according to the second example. Such an image is displayed on the display unit 30a. As illustrated in FIG. 14, for example, when the mode transitions to the Well-being mode, a home screen 430 as illustrated in the upper left is displayed on the display unit 30a. On the home screen 430, a video of the starry sky looked up from the forest is displayed as an example of the natural scenery. Furthermore, only minimum information such as time information may be displayed on the home screen 430. Next, in a case where it is determined that one or more users around the display unit 30a are eating and drinking by the detection of the context (For example, a case is assumed where one or more users intend to start eating and drinking in front of a television (a case where one or more users hold a chopstick or a glass).), the space production control unit 253 causes the video on the display unit 30a to transition to a video in a mode corresponding to the number of people. Specifically, for example, in the case of one person, the screen 432 in the one-person mode illustrated in the upper right of FIG. 14 is displayed. The screen 432 in the one-person mode may be, for example, a video of a bonfire. By looking at the bonfire, a relaxation effect can be expected. Note that, in the Well-being mode, a virtual world imitating one forest may be generated. Then, the screen transition may be performed such that a viewing direction in one forest seamlessly changes according to the detected context. For example, the home screen 430 in the Well-being mode displays an image of the sky seen from the forest. Next, when the context such as eating and drinking alone is detected, the line-of-sight (the direction of the virtual camera) directed toward the sky may be lowered, and the screen may be seamlessly transitioned to an angle of view of the bonfire video (screen 432) in the forest.

Furthermore, for example, in the case of a small number of people such as 2-3 people, the screen transitions to a small number mode screen 434 illustrated at the lower left of FIG. 14. The screen 434 in the small number mode may be, for example, a video with a little light in the depth of the forest. Even when a small number of people are eating and drinking, it is possible to produce a calm atmosphere that makes the people feel at ease. Note that screen transition from the one-person mode to the small number mode is also assumed. Also in this case, as an example, screen transition can be performed in which the direction (angle of view) viewed in one world view (for example, in the forest) seamlessly moves. Note that the number of people is 2-3 as an example of the small number, but the present example is not limited thereto, and 2 people may be a small number and 3 or more people may be a large number.

Furthermore, for example, in a case where there are a large number of users eating and drinking (for example, 4 or more users), the space production control unit 253 transitions to a screen 436 of a large number mode as illustrated in the lower right of FIG. 14. The screen 436 in the main mode may be, for example, a video in which bright light enters from the depth of the forest. It is possible to expect an effect of enlivening and enlivening the mood of the users.

The video for space production described above may be a moving image obtained by capturing an actual scene, may be a still image, or may be an image generated by 2D or 3D C G.

Furthermore, what kind of video is to be provided according to the number of people may be set in advance, or a video matching the atmosphere (character, liking, preference, and the like) of each user may be selected after each user is specified. Furthermore, since the provided video is intended to assist what the user is doing (for example, eating and drinking and conversation), it is preferable that explicit presentation such as a notification sound, a guide voice, or a message is not performed. The space production control can be expected to promote matters that are difficult for the user to notice, such as the user's emotion, mental state, and motivation, to a more preferable state.

Although only the video has been mainly described in FIG. 14, the space production control unit 253 can also perform production of sound and light together with presentation of the video. Furthermore, other examples of the information for performance include smell, wind, room temperature, humidity, smoke, and the like. The space production control unit 253 performs output control of these pieces of information using various output devices.

Subsequently, in a case where the number of people is two or more, the space production control unit 253 determines whether or not a cheer has been detected as the context (steps S331 and S340). Note that the context can be continuously performed. An operation such as cheering can also be detected from skeleton information and object information analyzed from the captured image. Specifically, for example, in a case where the position of the point of the wrist of the person holding the glass is above the position of the shoulder, the context such as making a cheer can be detected.

Next, in a case where a cheer is detected (step S331/Yes, S340/Yes), the space production control unit 253 performs control to capture a cheer scene by the camera 10a, store the captured image, and display the captured image on the display unit 30a (steps S334 and S343). FIG. 15 is a diagram for explaining imaging performed in response to a cheers action according to the second example. As illustrated in FIG. 15, when it is detected by analysis of the captured image of the camera 10a that a plurality of users (User A, User B, and User C) has performed cheers with glasses, the space production control unit 253 performs control to automatically capture a cheers scene by the camera 10a and display the captured image 438 on the display unit 30a. As a result, it is possible to provide more pleasant eating and drinking time to the users. The displayed image 438 disappears from the screen after a lapse of a predetermined time (for example, several seconds), and is saved in a predetermined storage area such as the storage unit 40.

When imaging a cheering scenery, the space production control unit 253 may output the shutter sound of the camera from the speaker 30b. Although not visible in FIG. 15, the speaker 30b can be arranged around the display unit 30a or the display unit 30a. Furthermore, the space production control unit 253 may appropriately control the lighting device 30c at the time of image-capturing so as to improve the picture appearance. Furthermore, here, as an example, it has been described that image-capturing is performed in the “cheers action”, but the present example is not limited thereto. For example, in a case where the user takes a certain pose with respect to the camera 10a, image-capturing may be performed. Furthermore, the present disclosure is not limited to imaging of a still image, and imaging of a moving image of several seconds or imaging of a moving image of several tens of seconds may be performed. When imaging is performed, a notification sound is output to clearly indicate to the user that imaging is being performed. Furthermore, an image may be captured in a case where it is detected that the user is excited from the volume, expression, or the like of the conversation. Furthermore, imaging may be performed at a preset timing. Furthermore, an image may be captured according to an explicit operation by the user.

Then, in a case where the number of persons holding the glasses has changed (step S346/Yes), the space production control unit 253 transitions to a mode according to the change (steps S323, S323, S326, S329, and S337). Here, the “number of persons holding glasses” is used, but the present disclosure is not limited thereto, and the “number of persons participating in a meal”, the “number of persons near a table”, or the like may be used. Furthermore, the screen transition can be performed seamlessly as described with reference to FIG. 14. Note that, in a case where the number of persons holding glasses or the like becomes zero, the screen returns to the home screen in the Well-being mode.

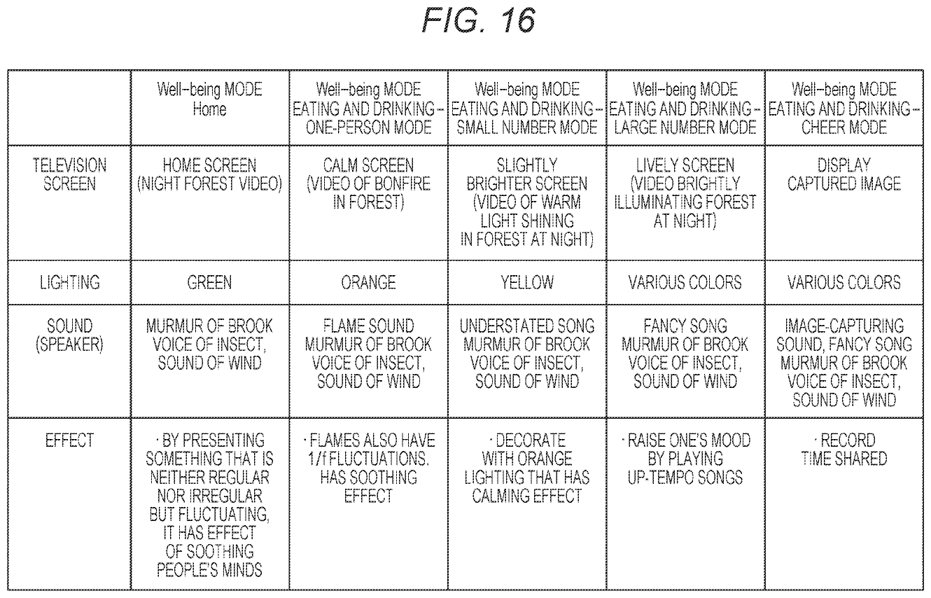

The example of the space production during eating and drinking has been described above. An example of various types of output control performed in the space production during eating and drinking is as illustrated in FIG. 16. FIG. 16 illustrates an example of what type of performance is to be performed in what state (context) and an effect exerted by the performance.

### 5-3. Modified Example

Next, a modified example of the second example will be described.

### 5-3-1. Heart Rate Reference

Space production with reference to a heart rate is also possible. For example, the analysis unit 251 can analyze the heart rate of the user on the basis of the captured image, and the space production control unit 253 can perform control to output appropriate music with reference to the context and the heart rate. The heart rate can be measured by a non-contact pulse wave detection technique for detecting a pulse wave from the color of the skin surface of the face image or the like.

For example, in a case where the context indicates that the user is resting alone, the space production control unit 253 may provide music of beats per minute (BPM) close to the heart rate of the user. Since the heart rate can change, music of BPM close to the heart rate of the user may be selected again when the next music is provided. By providing music of BPM close to the heart rate, it is expected to have a good effect on the user's mental state. Furthermore, since the tempo of the heart rate of a person is often synchronized with the tempo of the music to be listened to, it can be expected that a soothing effect is given by outputting music of BPM of the same level as the heart rate of a person at rest. As described above, the effect of healing the user can be exhibited not only from the video but also in music. Note that the measurement of the heart rate is not limited to the method based on the captured image of the camera 10a, and another dedicated device may be used.

Furthermore, in a case where it is indicated by the context that the users are having a conversation or eating and drinking in plural, the space production control unit 253 may provide music of beats per minute (BPM) corresponding to 1.0 times, 1.5 times, or 2.0 times the average value of the heart rate of each user. By providing music with a tempo faster than the current heart rate, it is possible to expect an effect of further raising excitement or enhancing mood. Note that, in a case where there is a user with an exceptionally fast heart rate among the plurality of users (a person who has run or the like), the user may be excluded, and the heart rates of the remaining users may be used. Furthermore, the measurement of the heart rate is not limited to the method based on the captured image of the camera 10a, and another dedicated device may be used.

Furthermore, even in a case where the user's actual preference for music is not known, music that is prepared in advance and generally preferred may be provided.

### 5-3-2. Further Encouraging Image-Capturing in Response to Cheers

Although it has been described that notification of the shutter sound is given the time of image-capturing corresponding to the above-described cheers action, the present disclosure is not limited thereto, and sound production up to image-capturing may be performed in order to further raise a cheer. For example, sound may be provided in accordance with the number of users. For example, in a case where there are three users, the scale may be assigned in the order in which the attitude for cheering (the position of the hand holding the glass rises above the position of the shoulder, or the like) can be detected, and a sound such as “Do, Mi, So” may be output. It is possible to give each user a recognition that he/she has taken a role by performing a cheers action, and it is possible to strengthen the meaning of being at the place. Furthermore, an upper limit of the number of people may be determined, and in a case where the number of people at the place is larger than the upper limit, sound may be made up to the upper limit number in the order of detection.

In this way, by performing a performance to raise a cheer, it is desired to raise a cheer, and it is possible to enhance the fun so that the fun of the cheer remains in memory as the fun of the party. The following control can also be performed as another way of making a sound at the time of toasting.
Every few minutes there will be a different cheer sound.
The sound is changed according to the color of the drink in the glass.
The sound is changed depending on which region of the angle of view of the camera the person making a cheer is in.

### 5-3-3. Production According to Excitement

In a case where there is a plurality of users, the context detection unit 252 may detect a degree of excitement as the context on the basis of the analysis result of the captured image and the collected sound data by the analysis unit 251, and the space production control unit 253 may perform space production according to the degree of excitement.

The degree of excitement can be detected, for example, by determining how much the users are looking at each other on the basis of a line-of-sight detection result of each user obtained from the captured image. For example, if four out of five people are looking at someone's face, it can be seen that they are absorbed in the talk. On the other hand, if all the five people do not face each other, it can be seen that the place is downhill.

Furthermore, the context detection unit 252 may detect the degree of excitement, for example, at a frequency of how many times laughter occurs per short time, on the basis of analysis of collected sound data (conversation sound or the like) collected by the microphone. Furthermore, the context detection unit 252 may determine that the user is excited in a case where the value of the change is a certain value or more on the basis of the analysis result of the change in the volume.

Furthermore, an example of presentation according to the degree of excitement by the space production control unit 253 will be described. For example, the space production control unit 253 may change the volume according to the change in the degree of excitement. Specifically, in a case where the user is excited, the space production control unit 253 may lower the volume of the music a little to make it easy to have a conversation, and in a case where the user is not excited, the space production control unit may raise the volume of the music a little (to an extent that it is not too noisy) so that a state (silent) in which the user does not have a conversation is not noticed. In this case, when someone starts a conversation, the volume is slowly lowered to the original volume.

Furthermore, the space production control unit 253 may perform production of providing a topic in a case where the degree of excitement decreases. For example, in a case where a cheers image has been captured, the space production control unit 253 may display the captured image on the display unit 30a together with the sound effect. As a result, the conversation can be naturally promoted. Furthermore, furthermore, space production control unit 253 may change the music while fade-in or fade-out when someone performs a specific gesture (for example, an operation of pouring a drink into a glass) while being in a heap. When the music changes, it can be expected to switch the mood. Note that the space production control unit 253 does not change the music even if the same gesture is performed again for a certain period of time after changing the music once.

Furthermore, the space production control unit 253 may change the video and the sound according to the degree of excitement. For example, while displaying the sky video, the space production control unit 253 may change the video to a sunny video in a case where the degree of excitement of a plurality of users becomes higher (than a predetermined value), and may change the video to a video with many clouds in a case where the degree of excitement becomes lower (than the predetermined value). Furthermore, in a case where the degree of excitement of a plurality of users becomes higher (than a predetermined value) during reproduction of a natural sound (murmur of brook, insect chirping, bird chirping, and the like), the space production control unit 253 may reduce the natural sound (for example, reduce four kinds of natural sounds to two kinds of sounds) (so as not to disturb the conversation), and may increase the natural sound (for example, increase three kinds of natural sounds to five kinds of sounds) in a case where the degree of excitement becomes lower (than the predetermined value) (so as silence is not noticed).

### 5-3-4. Perform Production when Pouring Drink into Glass

The space production control unit 253 may change the music according to the bottle poured into the glass. The bottle can be detected by analyzing object information based on the captured image. For example, the space production control unit 253 may recognize the color and shape of the bottle and the label of the bottle, and if the type and manufacturer are known, change the music to music corresponding to the type and manufacturer of the drink.

### 5-3-5. Change in Performance as Time Passes

The space production control unit 253 may change the performance according to the lapse of time. For example, in a case where the user is drinking alone, the space production control unit 253 may gradually reduce the fire of the bonfire (such as the image of the bonfire illustrated in FIG. 14) according to the lapse of time. Furthermore, the space production control unit 253 may change the color of the sky appearing in the video (from daytime to dusk, etc.), reduce the insect chirping, or reduce the volume according to the lapse of time. As described above, it is also possible to produce “end” by changing a video, music, or the like with the lapse of time.

### 5-3-6. Produce World View of Object Handled by User

For example, in a case where the user is reading a picture book to a child, the space production control unit 253 expresses the world view of the picture book with a video, music, lighting, and the like. Furthermore, the space production control unit 253 may change the video, the music, the lighting, and the like according to the scene change of the story every time the user turns the page. By detection of object information by analysis of a captured image, posture detection, and the like, it can be detected that the user is reading a picture book, what picture book the user is, turning a page, and the like. Furthermore, the context detection unit 252 can also grasp the content of the story and the scene change by voice analysis of voice data collected by the microphone. Furthermore, the space production control unit 253 can acquire information (view of the world, story) of the picture book from an external device such as a server by knowing what picture book the picture book is. Furthermore, the space production control unit 253 can estimate the progress of the story to some extent by acquiring the information of the story.

### 6. Third Example (Exercise Program Providing Function)

Next, as a third example, an exercise program providing function will be specifically described with reference to FIGS. 17 to 21. In the present example, when the user intends to actively exercise, an exercise program is generated and provided according to the ability of the user and the degree of interest in the exercise. The user can exercise with an exercise program suitable for the user without setting a level or an exercise load by the user. Providing an appropriate (not excessively loaded) exercise program for the user leads to continuation of exercise and improvement of motivation.

### 6-1. Configuration Example

FIG. 17 is a block diagram illustrating an example of a configuration of an information processing apparatus 1 that implements an exercise program providing function according to a third example. As illustrated in FIG. 17, the information processing apparatus 1 that implements the exercise program providing function includes a camera 10a, a control unit 20c, a display unit 30a, a speaker 30b, a lighting device 30c, and a storage unit 40. The camera 10a, the display unit 30a, the speaker 30b, the lighting device 30c, and the storage unit 40 are as described with reference to FIG. 3, and thus detailed description thereof is omitted here.

The control unit 20c functions as the exercise program providing unit 270. The exercise program providing unit 270 has functions of an analysis unit 271, a context detection unit 272, an exercise program generation unit 273, and an exercise program execution unit 274.

The analysis unit 271 analyzes the captured image acquired by the camera 10a, and detects skeleton information and object information. In the detection of the skeleton information, for example, each part (head, shoulder, hand, foot, and the like) of each person is recognized from the captured image, and the coordinate position of each part is calculated (acquisition of joint position). Furthermore, the detection of the skeleton information may be performed as posture estimation processing. Furthermore, in the detection of the object information, an object existing in the periphery is recognized. Furthermore, the analysis unit 271 can also integrate skeleton information and object information to recognize an object held in the hand of the user.

Furthermore, the analysis unit 271 may detect the face information from the captured image. The analysis unit 271 can specify the user by comparing the face information with the face information of each user registered in advance on the basis of the detected face information. The face information is, for example, information of feature points of the face. The analysis unit 271 compares the feature points of the face of the person analyzed from the captured image with the feature points of the face of one or more users registered in advance, and specifies a user having a matching feature (face recognition processing).

The context detection unit 272 detects the context on the basis of the analysis result of the analysis unit 271. More specifically, the context detection unit 272 detects the situation of the user as the context. In the present example, the context detection unit 272 detects that the user intends to actively exercise. At this time, the context detection unit 272 can detect what type of exercise the user intends to do from a change in posture of the user obtained by image analysis, clothes, a tool held in the hand, and the like. Note that the algorithm for context detection is not particularly limited. The context detection unit 272 may detect the context with reference to information such as posture, clothes, and belongings assumed in advance.

The exercise program generation unit 273 generates an exercise program suitable for the user with respect to the exercise that the user intends to perform according to the context detected by the context detecting unit 272. Various types of information for generating the exercise program may be stored in advance in the storage unit 40 or may be acquired from a server on a network.

Furthermore, the exercise program generation unit 273 generates the exercise program according to the ability and physical characteristics of the user in the exercise the user intends to perform and the interest degree of the use in the exercise the user intends to perform. The “ability of the user” can be determined, for example, from a level or a degree of improvement when the exercise is performed last time. Furthermore, the “physical feature” is a feature of the user's body, and examples thereof include information such as softness of the body, range of motion of a joint, presence or absence of injury, parts of the body that are difficult to move, and the like. In a case where there is a body part that is not desired to move or a body part that is difficult to move due to injury, disability, aging, or the like, an exercise program avoiding the part can be generated by registering in advance. Furthermore, the “degree of interest in the exercise” can be determined from the time or frequency of performing the exercise so far. The exercise program generation unit 273 generates an exercise program suitable for the level of the user that does not excessively load the user according to such ability and a degree of interest. Note that, in a case where the purpose of the exercise (adjustment of autonomic nerve, relaxation effect, improvement of stiff shoulder and back pain, elimination of insufficient exercise, improvement of metabolism, and the like) is input by the user, the exercise program may be generated in consideration of the purpose. In the generation of the exercise program, the contents, the number of exercises, the time, the order, and the like are assembled. The exercise program may be generated according to a predetermined generation algorithm, may be generated by combining predetermined patterns, or may be generated by using machine learning. For example, the exercise program generation unit 273 generates an exercise item list for each type of exercise (yoga, dance, stretching using tools, exercise, muscle strength training, pilates, jump rope, trampoline, golf, tennis, and the like). Specifically, an exercise program suitable for the user's ability, interest degree, purpose, and the like is generated on the basis of a database in which information such as skeleton information of the ideal posture, name, difficulty degree, effect, and consumed energy is associated.

The exercise program execution unit 274 controls predetermined video, audio, and lighting according to the generated exercise program. Furthermore, the exercise program execution unit 274 may appropriately feed back the posture and movement of the user acquired by the camera 10a to the screen of the display unit 30a. Furthermore, the exercise program execution unit 274 may display a video image as an example in accordance with the generated exercise program, and may explain tips and effects by text and voice, and may proceed to the next item when the user clears the explanation.

The configuration for realizing the exercise program providing function according to the present example has been specifically described above. Note that the configuration according to the present example is not limited to the example illustrated in FIG. 17. For example, the configuration for realizing the exercise program providing function may be realized by one device or may be realized by a plurality of devices. Furthermore, the control unit 20c, the camera 10a, the display unit 30a, the speaker 30b, and the lighting device 30c may be communicably connected to each other in a wireless or wired manner. Furthermore, at least one of the display unit 30a, the speaker 30b, or the lighting device 30c may be included. Furthermore, a configuration further including a microphone may be employed.

### 6-2. Operation Processing

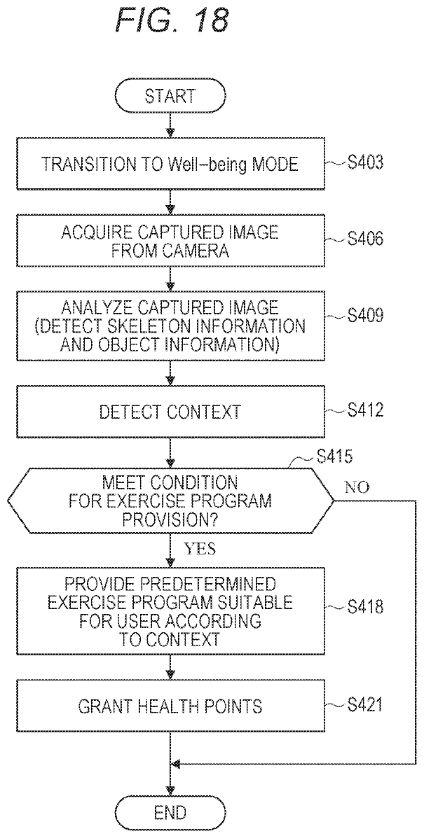

Next, operation processing according to the present example will be described with reference to FIG. 18. FIG. 18 is a flowchart illustrating an example of a flow of exercise program providing processing according to the third example.

As illustrated in FIG. 18, first, the control unit 20c transitions the operation mode of the information processing apparatus 1 from the content viewing mode to the Well-being mode (step S403). The transition to the Well-being mode is as described in step S106 of FIG. 4.

Next, a captured image is acquired by the camera 10a (step S406), and the analysis unit 271 analyzes the captured image (step S409). In the analysis of the captured image, for example, skeleton information and object information are detected.

Next, the context detection unit 272 detects the context on the basis of the analysis result (step S412).

Next, the exercise program providing unit 270 determines whether or not the detected context meets the condition for the exercise program provision (step S415). For example, in a case where the user intends to perform a predetermined exercise, the exercise program providing unit 270 determines that the condition is met.

Next, in a case where the detected context meets the condition (step S415/Yes), the exercise program providing unit 270 provides a predetermined exercise program suitable for the user according to the context (step S418). Specifically, the exercise program providing unit 270 generates a predetermined exercise program suitable for the user, and executes the generated exercise program.

Then, when the exercise program ends, the health point management unit 230 (See FIGS. 3 and 5) grants health points corresponding to the performed exercise program to the user (step S421).

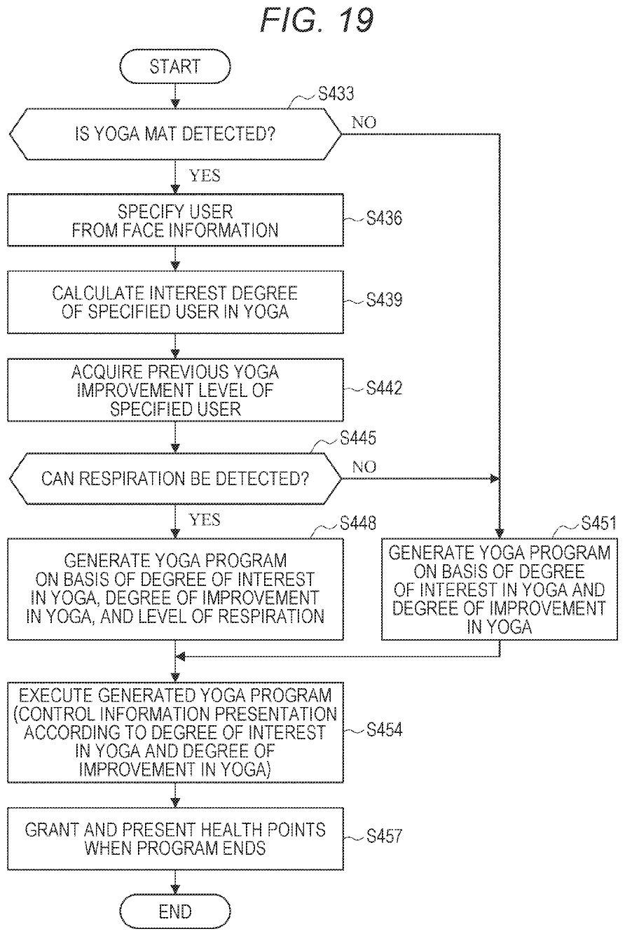

The flow of the exercise program providing process according to the present example has been described above. Note that the provision of the exercise program illustrated in step S418 described above will be further specifically described with reference to FIG. 19. In FIG. 19, a case of providing a yoga program will be described as a specific example.

FIG. 19 is a flowchart illustrating an example of a flow of yoga program providing processing according to the third example. This flow is performed in a case where the context is “the user is going to actively do yoga”.

As illustrated in FIG. 19, first, the context detection unit 272 determines whether or not the yoga mat is detected on the basis of the object detection based on the captured image (step S433). For example, in a case where the user appears in front of the display unit 30a with the yoga mat and places the yoga mat, the provision of the yoga program in the Well-being mode is started. Note that it may be assumed that an application (software) for providing the yoga program is stored in the information processing apparatus 1 in advance.

Next, the exercise program generation unit 273 specifies the user on the basis of the face information detected from the captured image by the analysis unit 271 (step S436), and calculates the interest degree of the specified user in yoga (step S439). For example, the interest degree of the user in yoga may be calculated on the basis of the use frequency and the use time of the yoga application of the user acquired from a database (the storage unit 40 or the like). For example, the exercise program generation unit 273 may set “no interest in yoga” in a case where the total use time of the yoga application in the last week is 0 minute, set “beginner's interest in yoga” in a case where the total use time is less than 10 minutes, set “intermediate interest in yoga” in a case where the total use time is 10 minutes or more and less than 40 minutes, and set “advanced interest in yoga” in a case where the total use time is 40 minutes or more.

Next, the exercise program generation unit 273 acquires the previous yoga improvement level (an example of ability) of the specified user (step S442). The information regarding the yoga application that the user has performed so far is accumulated in, for example, the storage unit 40 as the user information. The yoga improvement level is information indicating how much the user has reached, and can be granted by the system (the exercise program providing unit 270) in three stages of, for example, “Beginner level, Intermediate level, Advanced level” when the yoga program ends. The degree of yoga improvement can be granted on the basis of, for example, a difference between the ideal state (example) and the posture of the user, or an evaluation of the degree of swing of each point of the skeleton of the user.

Next, the analysis unit 271 detects respiration of the user (step S445). In yoga, since the effect of pose can be enhanced if the user is good at breathing, the ability of breathing is also treated as one of the ability of yoga of the user. Detection of respiration can be performed, for example, using a microphone. The microphone may be provided, for example, in a remote controller. Before starting the yoga program, the exercise program providing unit 270 prompts the user to take (a microphone provided in) the remote controller to his/her mouth to perform breathing and detects breathing. For example, the exercise program generation unit 273 sets the level of respiration to be advanced when the user inhales for 5 seconds and discharges for 5 seconds, sets the level to be intermediate level when the respiration is shallow, and sets the level to be beginner's level when the respiration stops in the middle. At this time, in a case where the user cannot breathe well, both the guide of the target value of respiration and the breathing result acquired from the microphone may be displayed and instructed.

Next, in a case where respiration can be detected (step S445/Yes), the exercise program generation unit 273 generates a yoga program suitable for the user on the basis of the interest degree of the specific user in yoga, the degree of improvement in yoga, and the level of respiration (step S448). Note that, in a case where the “purpose of doing yoga” is input by the user, the exercise program generation unit 273 may further generate the yoga program in consideration of the input purpose. Furthermore, the exercise program generation unit 273 may generate the yoga program using at least one of the interest degree of the specific user in yoga, the degree of improvement in yoga, or the level of respiration.

On the other hand, in a case where respiration cannot be detected (step S445/No), the exercise program generation unit 273 generates a yoga program suitable for the user on the basis of at least one of the interest degree of the specific user in yoga or the degree of improvement in yoga (step S451). In this case as well, in a case where the “purpose of doing yoga” is input by the user, the purpose may be considered.

Furthermore, here, as an example, it has been described that detection of respiration is performed in step S445, but the present example is not limited thereto, and detection of respiration may not be performed.

A specific example of generation of the yoga program will be described.

For example, in a case of a user having “an advanced degree of interest in yoga”, the exercise program generation unit 273 generates a program in which a pose with a high difficulty level is combined among poses suitable for the purpose input by the user. The difficulty level of each pose can be granted in advance by an expert.

Furthermore, for example, in the case of the user whose “the degree of interest in yoga is about beginner”, the exercise program generation unit 273 generates a program in which a pose with a low difficulty level is combined among poses suitable for the purpose input by the user. Furthermore, a pose in which the user has improved (has kept a posture close to an example for a certain period of time) in the yoga program up to the previous time may be replaced with a pose with a higher difficulty level. For example, even in the same type of pose, since the difficulty changes depending on the position where the hand is placed, the position of the foot, the bending state of the foot, and the like, the difficulty level of the pose as an example can be appropriately adjusted.

Furthermore, in a case where the user is determined to have “no degree of interest in yoga” due to elapse of one month or more from the previous execution of the yoga program, or the like, the exercise program generation unit 273 generates a yoga program that can reduce the number of poses to be usually assembled and easily give a sense of achievement. Moreover, in a case where the frequency of performing the yoga program is reduced or the user is not performing the yoga program for several months, or the like, the motivation of the user is lowered. Therefore, the exercise program generation unit 273 may lower the difficulty level and gradually raise the motivation by generating the yoga program with a small number of poses and around the poses that the user is good at in the yoga program so far.

The specific example of the generation of the yoga program has been described above. Note that the above-described specific examples are all examples, and the present example is not limited thereto.

Subsequently, the exercise program execution unit 274 executes the generated yoga program (step S454). In the yoga program, the video of the posture of the example by a guide (for example, CG) is displayed on the display unit 30a. The guide sequentially prompts the user to take each pose formed as the yoga program. As a rough flow, the guide first explains the effect of the pose, and then the guide shows an example of the pose. The user moves the body according to the example of the guide. Thereafter, there is a sign of the end of the pause, and the process proceeds to the description of the next pause. Then, when all the poses are finished, the yoga program end screen is displayed.

In order to assist the user's motivation during the yoga pose, the exercise program execution unit 274 may perform presentation according to the interest degree of the user in yoga or the degree of yoga improvement. For example, the exercise program execution unit 274 gives priority to advice regarding respiration so as to focus on respiration that is first important in yoga for the user having the “beginner level of yoga improvement”. A sucking timing and a discharging timing are presented by an audio guide and a text. Furthermore, the exercise program execution unit 274 may express the breathing timing on the screen so as to be intuitively easy to understand. For example, it may be expressed by a size of a body serving as a guide (inflate body when breathing in, and dent body when breathing out), or may be expressed by an arrow or a flow of air (effect) (effect heading to face may be displayed when breath is intake, and effect heading out from face may be displayed when breath is exhaled.). Furthermore, a circle may be superimposed and displayed on the guide and expressed by a change in the size of the circle (enlarging the circle when inhaling and reducing the circle when exhaling). Furthermore, a donut-shaped gauge graph may be superimposed and displayed as a guide and expressed by a change in the gauge graph (gradually increase graph when breathing in and gradually decrease graph when breathing out). Note that the information on the ideal breathing timing is registered in advance in association with each pose.

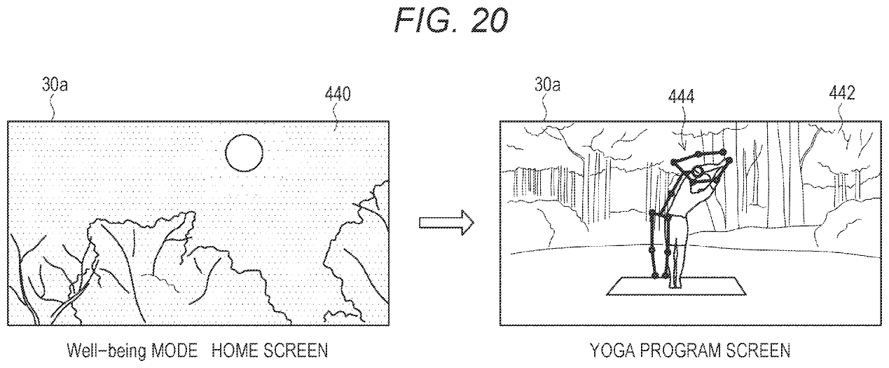

Furthermore, in the case of the user with the “beginner in the degree of yoga improvement”, the exercise program execution unit 274 may display the line connecting the points (joint positions) of the skeleton on the basis of the skeleton information of the user detected by the analysis of the captured image acquired by the camera 10a so as to overlap the person serving as the guide on the display screen of the display unit 30a. Here, FIG. 20 illustrates an example of a screen of a yoga program according to the present example. FIG. 20 illustrates a home screen 440 in a Well-being mode and a screen 442 of a yoga program that can be displayed thereafter. As illustrated in the screen 442 of the yoga program, the skeleton display 444 indicating the posture of the user detected in real time is superimposed and displayed on the video of the guide, so that even the beginner user can intuitively grasp how much the body should be tilted, how much the arm should be stretched, where the foot should be placed, and the like. Note that, in the example illustrated in FIG. 20, the posture of the user is expressed by a line segment, but the present example is not limited thereto. For example, the exercise program execution unit 274 may superimpose and display a semi-transparent silhouette (body silhouette) generated on the basis of the skeleton information on the guide. Furthermore, the exercise program execution unit 274 may express each line segment illustrated in FIG. 20 in a form in which some thickness is further added.

Furthermore, the exercise program execution unit 274 may present, in each pose, points to be conscious, such as which muscle should be consciously stretched and what should be noted, with a voice guide and characters in the case of a user with “intermediate degree of yoga improvement”. Furthermore, a matter to be a point, such as a direction of stretching the body, may be expressed using an arrow or an effect.

Furthermore, in the case of the user with the “advanced degree of yoga improvement”, the exercise program execution unit 274 reduces the amount of speech, characters, and effects presented by the guide as much as possible in order to concentrate on the “time to face oneself”, which is the original purpose of yoga. For example, description of the effect performed at the beginning of each pose may be omitted. Furthermore, presentation with priority given to space production may be performed so that the user can be immersed in the world view by reducing the volume of the voice of the guide and increasing the volume of the natural sound such as the voice of an insect and the murmur of a brook.

The specific example of the presentation method according to the degree of yoga improvement has been described above. Note that the exercise program execution unit 274 may change the method of presenting a guide when performing each pose according to the degree of improvement (previous) of each pose. Furthermore, the method of presenting guides in all poses may be changed in accordance with the interest degree of the user in yoga.

In this manner, by changing the presentation method in accordance with the degree of improvement in yoga or the degree of interest in yoga of the user, the matter (“breathing” for beginner, “point to be conscious (important point)” for intermediate) to be achieved by the user becomes clear, and the user can easily understand what to concentrate on. This makes it easier for the beginner or intermediate user to obtain a sense of achievement in each pose, in particular, than imitating a pose in a vague manner.

Furthermore, the exercise program execution unit 274 may perform guidance using surround sound. For example, in accordance with the guide “bend to the right”, sound of a stringer for matching sound or respiration of the guide may flow from the bending direction (right). Furthermore, depending on the pose, it may be difficult to see the display unit 30a during the pose. In the case of such a pose (in the case of a pose in which it is difficult to see the screen), the exercise program execution unit 274 may present a guide voice as if a guide character is coming to the feet (or near the head) of the user and talking by using surround sound. As a result, the user can feel realistic. Furthermore, the guide voice may be an advice (“Please raise your foot a little higher” or the like) corresponding to the posture of the user detected in real time.

Then, when all the poses are performed and the yoga program ends, the health point management unit 230 grants and presents the health points according to the yoga program (step S457).

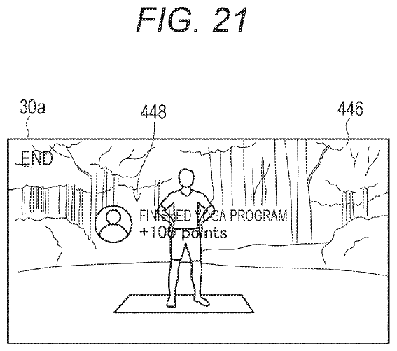

FIG. 21 is a diagram illustrating an example of a screen on which the health points granted to the user by the end of the yoga program is displayed. As illustrated in FIG. 21, for example, on the end screen 446 of the yoga program, a notification 448 indicating that the health points have been granted to the user may be displayed. The presentation of the health points may be more emphasized to display the health points particularly for the user who has performed the yoga program after a long time in order to lead to the next motivation.

Furthermore, when the yoga program is finished, the exercise program execution unit 274 may cause the guide to finally talk about the effect of moving the body or may compliment the user on the fact that the yoga program has been performed. Both can be expected to lead to the next motivation. Furthermore, the next motivation may be increased by performing guidance (new pose or the like) of the next yoga program such as “Let's take this pose in the next yoga program” for the user with the intermediate or advanced degree of interest in yoga. Furthermore, in a case where there is an item for which a pose has not been successfully taken in the yoga program performed this time, notification of the point of the last pose may be given.

Furthermore, in a case where the degree of improvement in posture has decreased as compared with a case where the user frequently (for example, one or more times a week) has performed a yoga program, negative feedback such as “the body has become hard” or “the body has wobbled” may be given to the user who performed the yoga program after a long time, and whose degree of interest in yoga has been intermediate or advanced in the past. When a negative feedback such as body wobble is given to a beginner user, motivation may be impaired, but in the case of a user who has been intermediate or advanced in the past, there is an effect of raising motivation by making the user realize that the user is in a bad state.

Furthermore, the exercise program execution unit 274 may display an image for comparing the face of the user imaged at the start of the yoga program and the face imaged at the end of the yoga program regardless of the degree of interest in yoga or the like. At this time, it is possible to give the user a sense of accomplishment by conveying the effect of performing the yoga program such as “Blood flow has improved” by the guide.

Furthermore, at the end of the yoga program, the exercise program providing unit 270 may calculate the degree of yoga improvement of the user on the basis of the result of the current yoga program (the degree of achievement of each pose, etc.) and newly register the degree of yoga improvement as the user information. Furthermore, the exercise program providing unit 270 may calculate the degree of improvement in each pose during the execution of the yoga program and store the degree of improvement as the user information. The degree of improvement in each pose may be evaluated on the basis of, for example, a difference between the state of the skeleton of the user during the pose and the ideal skeleton state, the degree of swing of each point of the skeleton, or the like. Furthermore, the exercise program providing unit 270 may calculate the degree of improvement in “respiration”. For example, at the end of the yoga program, the user may be instructed to perform breathing on (the remote controller provided with) the microphone, and the breathing information may be acquired to calculate the degree of improvement. In a case where the user cannot breathe well, the exercise program providing unit 270 may display both the guide of the target value of respiration and the respiration result acquired from the microphone. Furthermore, in a case where the user has performed the yoga program after a long time and it is detected that the respiration has become shallow during the yoga program, the exercise program providing unit 270 may perform feedback such as “the respiration has become shallower than the previous time” at the end of the yoga program. Furthermore, as another acquisition method of the degree of yoga improvement, it is also assumed that data received from a sensor provided in the wear of the stretch material worn by the user is used.

After the end of the yoga program, the screen of the display unit 30a returns to the home screen in the Well-being mode.

The operation processing of the third example has been specifically described above. Note that each step of the operation processing illustrated in FIG. 19 may be appropriately skipped, processed in parallel, or processed in the reverse order.

### 6-3. Modified Example

The exercise program generation unit 273 may further incorporate the user's lifestyle when generating an exercise program suitable for the user. For example, in view of the time when the yoga program is started and the tendency of the lifestyle of the user, a shorter program configuration may be used when the bedtime is approaching and there is no time. Furthermore, the program configuration may be changed according to the time zone in which the yoga program is started. For example, in a case where the sleeping time is close, it is important to suppress the action of the sympathetic nerve, and thus, it is possible to generate a program that makes the user conscious of breathing more slowly than usual in the forward-bending pose without adopting the back-bending pose (promoting the action of the sympathetic nerve).

Furthermore, when generating an exercise program suitable for the user, the exercise program generation unit 273 may further consider the interest degree of the user in the exercise determined by the exercise interest degree determination unit 234 on the basis of the health points of the user.

Furthermore, when the health point management unit 230 notifies the user of the granting of the health points, the exercise program providing unit 270 may also give a proposal of “Would you like to move your body in a yoga program?” to a user who has a high degree of interest in exercise but has never performed a specific exercise program (for example, a yoga program).

### 7. Supplement

The preferred embodiment of the present disclosure has been described above in detail with reference to the accompanying drawings, but the present technology is not limited to such examples. It is obvious that those with ordinary skill in the technical field of the present disclosure may conceive various modifications or corrections within the scope of the technical idea recited in claims, and it is naturally understood that they also fall within the technical scope of the present disclosure. Furthermore, it is also possible to create one or more computer programs for causing hardware such as the CPU, the ROM, and the RAM built in the information processing apparatus 1 described above to exhibit the functions of the information processing apparatus 1. Furthermore, a computer-readable storage medium that stores the one or more computer programs is also provided.

Furthermore, the effects described in the present specification are merely exemplary or illustrative, and not restrictive. That is, the technology according to an embodiment of the present disclosure can exhibit other effects apparent to those skilled in the art from the description of the present specification, in addition to the effects described above or instead of the effects described above.

Note that the present technology can also have the following configuration.

(1)

An information processing apparatus including
a control unit that performs:
a process of recognizing a user existing in a space on the basis of a detection result of a sensor disposed in the space and calculating health points indicating that a healthy behavior has been performed from an action of the user; and a process of giving notification of the health points.
   (2)

The information processing apparatus according to (1), in which the sensor is a camera, and
the control unit analyzes a captured image that is the detection result, and when determining that the user is performing a predetermined posture or movement registered in advance as a healthful behavior from a posture or movement of the user, the control unit grants health points corresponding to the behavior to the user.
   (3)

The information processing apparatus according to (2), in which the control unit calculates the health points to be granted to the user according to a difficulty level of the behavior.

(4)

The information processing apparatus according to any one of (1) to (3), in which the control unit stores information on the health points granted to the user in a storage unit, and performs control to give notification of a total of the health points of the user in a certain period at a predetermined timing.

(5)

The information processing apparatus according to any one of (1) to (4), in which the sensor is provided in a display device installed in the space, and detects information regarding one or more persons acting around the display device.

(6)

The information processing apparatus according to (5), in which the control unit performs control to give notification on the display device that the health points have been granted.

(7)

The information processing apparatus according to (6), in which the control unit analyzes a situation of one or more persons existing around the display device on the basis of the detection result, and performs control to give notification by displaying information on health points of the user on the display device at a timing when the situation satisfies a condition.

(8)

The information processing apparatus according to (7), in which the situation includes a degree of concentration in viewing of content reproduced on the display device.

(9)

The information processing apparatus according to any one of (1) to (8), in which the control unit calculates an interest degree of the user in exercise on the basis of a total of the health points in a certain period or a temporal change of the total.

(10)

The information processing apparatus according to (9), in which the control unit determines contents of the notification according to a degree of interest in the exercise.

(11)

The information processing apparatus according to (10), in which the contents of the notification includes information regarding health points granted this time, a reason for granting, and a recommended stretch.

(12)

The information processing apparatus according to any one of (1) to (11), in which the control unit acquires a situation of one or more persons existing in the space on the basis of the detection result, and performs control to output a video, an audio, or lighting for space production according to the situation from one or more output devices installed in the space.

(13)

The information processing apparatus according to (12) described above, in which the situation includes at least any of a number of persons, an object held in a hand, things being performed, a state of biometric information, an excitement degree, or a gesture.

(14)

The information processing apparatus according to (12) or (13), in which when an operation mode of a display device installed in the space and used for viewing content transitions to a mode for providing a function for promoting a good life, the control unit starts output control for the space production according to the detection result.

(15)

The information processing apparatus according to any one of (1) to (14), in which
the control unit performs:
a process of determining an exercise that the user intends to perform on the basis of the detection result;
a process of individually generating an exercise program of the determined exercise according to information of the user; and
a process of presenting the generated exercise program on a display device installed in the space.
   (16)

The information processing apparatus according to (15), in which the control unit grants the health points to the user after an end of the exercise program.

(17)

The information processing apparatus according to (15) or (16), in which when an operation mode of a display device installed in the space and used for viewing content transitions to a mode for providing a function for promoting a good life, the control unit starts presentation control of the exercise program according to the detection result.

(18)

An information processing method in which a processor including:
recognizing a user existing in a space on the basis of a detection result of a sensor disposed in the space and calculating health points indicating that a healthy behavior has been performed from an action of the user; and
giving notification of the health points.
   (19)

A program for causing a computer to function as a control unit that performs:
a process of recognizing a user existing in a space on the basis of a detection result of a sensor disposed in the space and calculating health points indicating that a healthy behavior has been performed from an action of the user; and
a process of giving notification of the health points.

### REFERENCE SIGNS LIST

1 Information processing apparatus
10 Input unit
10a Camera
20 (20a to 20c) Control unit
210 Content viewing control unit
230 Health point management unit
250 Space production unit
270 Exercise program providing unit
30 Output unit
30a Display unit
30b Speaker
30c Lighting device
40 Storage unit

1. An information processing apparatus comprising
a control unit that performs:
a process of recognizing a user existing in a space on a basis of a detection result of a sensor disposed in the space and calculating health points indicating that a healthy behavior has been performed from an action of the user; and
a process of giving notification of the health points.

2. The information processing apparatus according to claim 1, wherein
the sensor is a camera, and
the control unit analyzes a captured image that is the detection result, and when determining that the user is performing a predetermined posture or movement registered in advance as a healthful behavior from a posture or movement of the user, the control unit grants health points corresponding to the behavior to the user.

3. The information processing apparatus according to claim 2, wherein the control unit calculates the health points to be granted to the user according to a difficulty level of the behavior.

4. The information processing apparatus according to claim 1, wherein the control unit stores information on the health points granted to the user in a storage unit, and performs control to give notification of a total of the health points of the user in a certain period at a predetermined timing.

5. The information processing apparatus according to claim 1, wherein the sensor is provided in a display device installed in the space, and detects information regarding one or more persons acting around the display device.

6. The information processing apparatus according to claim 5, wherein the control unit performs control to give notification on the display device that the health points have been granted.

7. The information processing apparatus according to claim 6, wherein the control unit analyzes a situation of one or more persons existing around the display device on a basis of the detection result, and performs control to give notification by displaying information on health points of the user on the display device at a timing when the situation satisfies a condition.

8. The information processing apparatus according to claim 7, wherein the situation includes a degree of concentration in viewing of content reproduced on the display device.

9. The information processing apparatus according to claim 1, wherein the control unit calculates an interest degree of the user in exercise on a basis of a total of the health points in a certain period or a temporal change of the total.

10. The information processing apparatus according to claim 9, wherein the control unit determines contents of the notification according to a degree of interest in the exercise.

11. The information processing apparatus according to claim 10, wherein the contents of the notification includes information regarding health points granted this time, a reason for granting, and a recommended stretch.

12. The information processing apparatus according to claim 1, wherein the control unit acquires a situation of one or more persons existing in the space on a basis of the detection result, and performs control to output a video, an audio, or lighting for space production according to the situation from one or more output devices installed in the space.

13. The information processing apparatus according to claim 12, wherein the situation includes at least any of a number of persons, an object held in a hand, things being performed, a state of biometric information, an excitement degree, or a gesture.

14. The information processing apparatus according to claim 12, wherein when an operation mode of a display device installed in the space and used for viewing content transitions to a mode for providing a function for promoting a good life, the control unit starts output control for the space production according to the detection result.

15. The information processing apparatus according to claim 1, wherein
the control unit performs:
a process of determining an exercise that the user intends to perform on a basis of the detection result;
a process of individually generating an exercise program of the determined exercise according to information of the user; and
a process of presenting the generated exercise program on a display device installed in the space.

16. The information processing apparatus according to claim 15, wherein the control unit grants the health points to the user after an end of the exercise program.

17. The information processing apparatus according to claim 15, wherein when an operation mode of a display device installed in the space and used for viewing content transitions to a mode for providing a function for promoting a good life, the control unit starts presentation control of the exercise program according to the detection result.

18. An information processing method in which a processor comprising:
recognizing a user existing in a space on a basis of a detection result of a sensor disposed in the space and calculating health points indicating that a healthy behavior has been performed from an action of the user; and
giving notification of the health points.

19. A program for causing a computer to function as a control unit that performs:
a process of recognizing a user existing in a space on a basis of a detection result of a sensor disposed in the space and calculating health points indicating that a healthy behavior has been performed from an action of the user; and
a process of giving notification of the health points.
